(19) United States
(12) Patent Application Publication
(10) Pub. No.: US 2023/0338344 A1
(43) Pub. Date: Oct. 26, 2023

(54) AMPK Activators and Methods of Use Thereof

(71) Applicant: Bioverativ Therapeutics Inc., Waltham, MA (US)

(72) Inventors: Pauline RIMMELÉ, Bridgewater, NJ (US); Dieter SCHMOLL, Frankfurt am Main (DE); Yannis HARA, Bridgewater, NJ (US)

(73) Assignee: Bioverativ Therapeutics Inc., Waltham, MA (US)

(21) Appl. No.: 18/191,293

(22) Filed: Mar. 28, 2023

Related U.S. Application Data
(63) Continuation of application No. PCT/US2021/052512, filed on Sep. 29, 2021.
(60) Provisional application No. 63/212,343, filed on Jun. 18, 2021.
(60) Provisional application No. 63/085,179, filed on Sep. 30, 2020.

(51) Int. Cl. A61K 31/4365; A61K 31/404; A61K 31/17; A61P 7/06

(52) U.S. Cl. CPC A61K 31/4365; A61K 31/404; A61K 31/17; A61P 7/06

(57) ABSTRACT

This disclosure to methods for treating or preventing particular symptoms and disorders which are associated with blood disorders using AMPK activators. Also disclosed are pharmaceutical composition comprising an AMPK activator for use in said methods.

This patent application is a continuation of International Application No. PCT/US2021/052512, filed Sep. 29, 2021, which claims priority to US Provisional Patent Application No. 63/085,179, filed Sep. 30, 2020; and U.S. Provisional Patent Application No. 63/212,343, filed Jun. 18, 2021, the contents of each of which are incorporated herein by reference in their entirety for all purposes.

Disclosed herein are compounds that activate 5′-adenosine monophosphate-activated protein kinase (AMPK), pharmaceutical compositions containing these compounds, and methods for treating or preventing blood disorders such as hemoglobinopathies.

### BACKGROUND

The 5′AMP-activated protein kinases (AMPK) are sensors of the overall energy level in mammalian cells and organs. AMPK is activated by an increase in the intracellular AMP/ATP ratio, induced for example by a metabolic stress, hormones, or nutrient signaling pathways (Viollet et al. Crit Rev Biochem Mol Biol, 2010. 45(4): p. 276-95). When activated, AMPK blocks the metabolic pathways which consume ATP (such as fatty acid synthesis in adipocytes, cholesterol synthesis in the liver, and insulin secretion in s-cells) and activates the metabolic pathways which produce ATP (such as fatty acid absorption and beta-oxidation in various tissues, glycolysis in the heart, and the biogenesis of mitochondria in skeletal muscle). AMPK also modulates the transcription of genes which participate in energy metabolism, exerting a metabolic control to facilitate energy (Viollet et al.). Moreover, AMPK participates in the regulation of non-metabolic processes such as cell growth, progression of the cell cycle, and organization of the cytoskeleton (Williams T., et al., Proc Natl Acad Sci USA, 2011. 108(14): p. 5849-54). Although the activation of AMPK is an adaptive response to an energy stress in many biological systems, AMPK plays an important role in maintaining physiological functions and for adaptation to pathophysiological conditions. Furthermore, it has been reported that AMPK promotes anti-inflammatory effects in vitro in murine and human macrophages by promoting macrophage polarization to an anti-inflammatory functional phenotype (Sag, D., et al., J Immunol, 2008. 181(12): pp. 8633-41). AMPK is an αβγ trimer of three subunits comprised of twelve known isoforms that are based on all possible combinations of 2α, 2β and 3γ subunits. Activators of AMPK, including 5′AMP and pharmacological small molecule AMPK activators, bind the CBS sites in the γ subunits and the ADAM site, or “allosteric drug and metabolite” binding site, that lies between the α and β subunits (Aledavood et al., J Chem Inf Model, 2019. 59(6): p. 2859-2870). AMPK can be activated both by activator binding to the ADAM site and by phosphorylation of Thr172 of the α-subunit. Pharmacological activators of AMPK binding to the ADAM site are known to selectively activate AMPK isoforms containing the β1-subunit (β1-selective AMPK activators or selective β1-AMPK activators) or containing either a β1-subunit or a β2-subunit (pan-selective AMPK activators).

Sickle cell disease (SCD), also called sickle cell anemia, is one of the most common monogenic diseases, with 330,000 affected individuals born annually worldwide (Piel, F. B., et al., Lancet, 2013. 381(9861): p. 142-51). It is the result of substitution of glutamic acid by valine at position 6 of the β-globin subunit of hemoglobin. The presence of this mutant β-globin subunit leads to the production of abnormal hemoglobin S (HbS) that polymerizes in red blood cells under conditions of low oxygen, which affects blood flow rheology and ultimately leads to vaso-occlusive crises and end organ damage (Barabino et al. Annu Rev Biomed Eng, 2010. 12: p. 345-67). Oxidative stress in erythrocytes is also enhanced and causes haemolysis (Vona, R., et al., Antioxidants (Basel), 2021. 10(2), and inflammation involving monocytes and proinflammatory macrophages is exacerbated in tissues and blood and participates to the organ damage process (Van Beers, E. J., et al., Circ Res, 2015. 116(2): p. 298-306; and Allah, S., et al., Haematologica, 2020. 105(2): p. 273-283).

It has been reported that increased fetal hemoglobin (HbF), whether endogenous or drug induced, ameliorates the symptoms and complications of SCD by preventing red blood cell sickling through dilution of the concentration of HbS in erythrocytes and inhibiting the polymerization of HbS (Piel et al. Lancet, 2013. 381(9861): p. 142-51). Although hydroxyurea (HU) is the only US Food and Drug Administration approved HbF-inducing agent for use in adults with SCD (Steinberg, M. H., et al., JAMA, 2003. 289(13): p. 1645-51), up to 50% of patients do not experience clinical improvement on HU. This variability of HbF-inducing response to HU is still under investigation (Platt, et al., N Engl J Med, 2008. 358(13): p. 1362-9). Increasing HbF in erythrocytes is also beneficial to treat clinical manifestations of other β-hemoglobinopathies, including β-thalassemia (Ye, L., et al., Proc Natl Acad Sci USA, 2016. 113(38): p. 10661-5). Finally, decreasing inflammation and oxidative stress in SCD leads to beneficial clinical effects in patients (Kato, G. J., et al., Nat Rev Dis Primers, 2018. 4: p. 18010).

FOXO3 is the transcription factor forkhead box 0-3 hypothesized to be responsible for increasing HbF when overexpressed or activated in CD34+ erythroid cells (Zhang, Y., et al., Blood, 2018. 132(3): p. 321-333). FOXO3 is in the forkhead/winged helix box gene, group O (FoxO) family of proteins that are evolutionarily conserved transcription factors. FOXO transcription factors integrate many important cellular functions and were originally identified as regulators of insulin-related genes. FOXO transcription factors regulate genes involved in biological processes, including substrate and energy metabolism, protein turnover, cell survival, oxidative stress, proteostasis, apoptosis and cell death, cell cycle regulation, metabolic processes, immunity, inflammation and stem cell maintenance (Morris, B. J., et al., Gerontology, 2015. 61(6): p. 515-25; and Stefanetti, R. J., et al., F1000Res, 2018. 7). In particular, FOXO3 is required for the maintenance of murine hematopoietic stem cells and functions (Yalcin, S., et al., J Biol Chem, 2008. 283(37): p. 25692-25705; and Rimmele, P., et al., EMBO Rep, 2015. 16(9): p. 1164-76).

In addition, FOXO3 is a key mediator of erythroid terminal maturation, regulating cell cycle in early stages of erythropoiesis and critical for ROS regulation, enucleation and mitochondrial clearance in late stages (Marinkovic, D., et al., J Clin Invest, 2007. 117(8): p. 2133-44; and Liang, R., et al., PLoS Genet, 2015. 11(10): p. e1005526). In this context modulation of FOXO3 might influence erythroid disorders as has been reported specifically for hemoglobinopathies (Zhang, Y., et al., Blood, 2018. 132(3): p. 321-333; Pourfarzad, F., et al., Cell Rep, 2013. 4(3): p. 589-600; and Franco, S. S., et al., 2014. 99(2): p. 267-75). Sirtuin 1 (Sirt1) is a homolog of the NAD-dependent protein silent information regulator 2 in yeast and is a deacetylase for histones and other proteins. Deacetylation of FOXO3 by Sirt1 is an important regulatory control mechanism of FOXO3 in many cells and tissues (Giannakou, M. E., et al., Science, 2004. 305(5682): p. 361). Increased acetylation of FOXO3 by elimination of the deacetylase Sirt1 in hematopoietic stem cells (HSC) results in a defective lineage specification biased toward the myeloid lineage and a phenotype that resembles aged HSC (Rimmele, P., et al., Stem Cell Reports. 2014 Jul. 8; 3(1): 44-59).

An in vitro study in differentiating human CD34+ erythroid cells showed that decreasing FOXO3 by shRNA delivery decreased expression of HbF, and conversely, over-expression of FOXO3 increased HbF expression in these cells. It was theorized that AMPK phosphorylates and activates FOXO3, and metformin was used to increase cellular AMP and indirectly activate AMPK to increase HbF expression (Zhang, Y., et al., Blood, 2018. 132(3): p. 321-333).

New HbF inducing drugs are therefore urgently needed. Surprisingly we found and demonstrated herein below that β1-selective AMPK activators and pan-AMPK activators increase expression of HbF in differentiating human lineage erythroid cells without affecting terminal differentiation as measured by enucleation. Moreover, isoform β1 is predominant in erythroid lineage in human bone marrow compared to β2, making β1-selective AMPK activators more specific to the erythroid lineage compared to pan-AMPK activators that potentially activate other cells lineage or tissues expressive β2. In support of the benefit of β1-selective AMPK activation to induce HbF in erythroid lineage, we show that a β1-selective AMPK activator does not affect terminal differentiation as measured by enucleation and does not cause anemia in vivo. Furthermore, a β1-selective AMPK activator is shown to cause anti-inflammatory responses by promoting macrophages polarization and differentiation to an anti-inflammatory M1 phenotype, potentially resulting in a decrease of the inflammatory response which is a key component of the sickle cell disease pathophysiology. Finally, we show activation of the nrf2-oxidative stress response by activating AMPK in human CD34 positive cells, potentially protecting against oxidative stress occurring in sickle cell disease.

### SUMMARY OF THE INVENTION

In one aspect, the present application relates to a method of treating hemoglobinopathies, the method comprising administering an effective amount of a selective 5′-adenosine monophosphate-activated protein kinase (AMPK) activator to a patient in need thereof. In some embodiments, the hemoglobinopathy is selected from β-thalassemia and sickle cell disease (SCD).

In some embodiments, the AMPK activator is a β1-selective AMPK activator that is a more selective activator of β1-AMPK versus activation of β2-AMPK or of activation of both β1-AMPK and β2-AMPK (pan-activation). In some embodiments, the β1-selective AMPK activator may possess at least about a 10-fold selective activation, at least about a 50-fold selective activation, at least about a 100-fold activation, at least about a 300-fold selective activation, or at least about a 500 fold selective activation for β1-AMPK relative to β2-AMPK.

In some embodiments, the β1-selective AMPK activator has an EC₅₀ (half-maximal concentration required for full activation) for the activation of β1-AMPK of about 100 nM or less. In other embodiments, the β1-selective AMPK activator has an EC₅₀ of about 50 nM or less for the activation of β1-AMPK. On other embodiments, the β1-selective AMPK activator has an EC₅₀ of about 10 nM or less for the activation of β1-AMPK. EC₅₀ values may be determined according to the enzymatic assays performed in Schmoll et al. (Hepatol Commun, 2020; Vol. 4, No. 7, pp. 1056-1072).

In some embodiments, the β1-selective AMPK activator increases the activity of AMPK above the baseline activity (as measured by autophosphorylation of the α-subunit of AMPK or phosphorylation of a peptide or protein substrate) by 50% or more. In other embodiments, the β1-selective AMPK activator increases the activity of AMPK above the baseline activity by 100% or more. In other embodiments, the β1-selective AMPK activator increases the activity of AMPK above the baseline activity by 150% or more.

In some embodiments, the β1-selective AMPK activator is a compound of Formula (I):

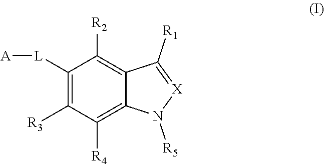

or a pharmaceutically acceptable salt thereof, wherein
X is N or CH;
R₁ is —C(O)OR_{A}, —C(O)NR_{B}R_{C}, —S(O₂)OR_{A}, —S(O₂)NHC(O)R_{D},
5-oxo-4,5-dihydro-1,2,4-oxadiazol-3-yl, or 1H-tetrazol-5-yl;
R_{A} is H or (C₁-C₆)alkyl;
R_{B} and R_{C} are independently H, (C₁-C₆)alkyl, or —S(O₂)R_{D};
R_{D} is (C₁-C₆)alkyl, —CF₃, or phenyl, wherein the phenyl is optionally substituted with 1, 2, 3, 4, or 5 substituents that are independently (C₁-C₆)alkoxy, (C₁-C₆)alkyl, cyano, halogen, halo(C₁-C₆)alkoxy, halo(C₁-C₆)alkyl, hydroxy, mercapto, nitro, or NR_{E}R_{F};
R_{E} and R_{F} are independently H or (C₁-C₆)alkyl;
R₂, R₃, and R₄ are independently H, (C₁-C₆)alkoxy, (C₁-C₆)alkyl, (C₁-C₆)alkylthio, carboxy, cyano, halogen, halo(C₁-C₆)alkoxy, halo(C₁-C₆)alkyl, hydroxy,hydroxy(C₁-C₈)alkyl, mercapto, nitro, —NR_{G}R_{H} or (NR_{G}R_{H})carbonyl;
R_{G} and R_{H} are independently H, (C₁-C₆)alkyl, or (C₁-C₆)alkylcarbonyl;
R₅ is H or (C₁-C₆)alkyl;
L is a bond, O, S, NRA, (C₁-C₆)alkylene, (C₂-C₆)alkenylene, or (C₂-C₆)alkynylene; A is phenyl, 2,3-dihydrobenzo[b][1,4]dioxinyl, 2,3-dihydrobenzofuranyl, 2,3-dihydro-1H-indenyl, imidazolyl, pyrazinyl, pyrazolyl, pyridinyl, pyrimidinyl, or thiazolyl, wherein each is optionally substituted with 1, 2, 3, 4, or 5 substituents that are independently (C₁-C₆)alkoxy, (C₁-C₆)alkoxy(C₁-C₆)alkoxy, (C₁-C₆)alkoxy(C₁-C₆)alkyl, (C₁-C₆)alkoxycarbonyl, (C₁-C₆)alkyl, (C₁-C₆)alkylcarbonyl, (C₁-C₆)alkylthio, aryl, aryl(C₁-C₆)alkoxy, aryl(C₁-C₆)alkyl, arylcarbonyl, aryloxy, carboxy, carboxy(C₁-C₆)alkoxy, carboxy(C₁-C₆)alkyl, cyano, (C₃-C₈)cycloalkyl, (C₃-C₈)cycloalkyl(C₁-C₆)alkoxy, (C₃-C₈)cycloalkyl(C₁-C₆)alkyl, (C₃-C₈)cycloalkylcarbonyl, (C₃-C₈)cycloalkyloxy, halogen, halo(C₁-C₆)alkoxy, halo(C₁-C₆)alkyl, heteroaryl, heteroaryl(C₁-C₆)alkoxy, heteroaryl(C₁-C₆)alkyl, heteroarylcarbonyl, heteroaryloxy, (C₃-C₆)heterocycle, (C₃-C₇)heterocycle(C₁-C₆)alkoxy, (C₃-C₇)heterocycle(C₁-C₆)alkyl, (C₃-C₇)heterocyclocarbonyl, (C₃-C₇)heterocyclecarbonyl(C₁-C₆)alkyl, (C₃-C₇)heterocycleoxy, hydroxy, hydroxy(C₁-C₆)alkoxy, hydroxy(C₁-C₆)alkyl, mercapto, nitro, —NR_{J}R_{K}, (NR_{J}R_{K})carbonyl, —NR_{M}R_{N}, —NR_{M}R_{N}(C₁-C₆)alkoxy, (NR_{M}R_{N})carbonyl, (NR_{M}R_{N})carbonyl(C₁-C₆)alkyl, or (NR_{M}R_{N})carbonyl(C₁-C₆)alkoxy; wherein the aryl, aryl(C₁-C₆)alkoxy, aryl(C₁-C₆)alkyl, arylcarbonyl, and aryloxy are optionally substituted with 1, 2, 3, 4, or 5 substituents that are independently (C₁-C₆)alkoxy, (C₁-C₆)alkoxycarbonyl, (C₁-C₆)alkyl, (C₁-C₆)alkylcarbonyl, (C₁-C₆)alkylthio, carboxy, cyano, halogen, halo(C₁-C₆)alkoxy, halo(C₁-C₆)alkyl, hydroxy, hydroxy(C₁-C₆)alkyl, mercapto, nitro, —NR_{M}R_{N}, or (NR_{M}R_{N})carbonyl; wherein the halo(C₁-C₆)alkyl is optionally substituted with 1 or 2 hydroxy groups; wherein the (C₃-C₈)cycloalkyl, (C₃-C₈)cycloalkyl(C₁-C₆)alkoxy, (C₃-C₈)cycloalkyl(C₁-C₆)alkyl, (C₃-C₈)cycloalkylcarbonyl, and (C₃-C₈)cycloalkyloxy are optionally substituted with 1, 2, or 3 substituents that are independently (C₁-C₆)alkoxy, (C₁-C₆)alkoxycarbonyl, (C₁-C₆)alkyl, (C₁-C₆)alkylcarbonyl, (C₁-C₆)alkylthio, carboxy, cyano, halogen, halo(C₁-C₆)alkoxy, halo(C₁-C₆)alkyl, hydroxy, hydroxy(C₁-C₆)alkyl, mercapto, nitro, —NR_{M}R_{N}, or (NR_{M}R_{N})carbonyl; wherein the heteroaryl, heteroaryl(C₁-C₆)alkoxy, heteroaryl(C₁-C₆)alkyl, heteroarylcarbonyl, and heteroaryloxy, are optionally substituted with 1, 2, or 3 substituents that are independently (C₁-C₆)alkoxy, (C₁-C₆)alkoxycarbonyl, (C₁-C₆)alkyl, (C₁-C₆)alkylcarbonyl, (C₁-C₆)alkylthio, carboxy, cyano, halogen, halo(C₁-C₆)alkoxy, halo(C₁-C₆)alkyl, hydroxy, hydroxy(C₁-C₆)alkyl, mercapto, nitro, —NR_{M}R_{N}, or (NR_{M}R_{N})carbonyl; and wherein the (C₃-C₇)heterocycle, (C₃-C₇)heterocycle(C₁-C₆)alkoxy, (C₃-C₇)heterocycle(C₁-C₆)alkyl, (C₃-C₇)heterocyclecarbonyl, (C₃-C₇)heterocyclecarbonyl(C₁-C₆)alkyl, and
(C₃-C₇)heterocycleoxy, are optionally substituted with 1, 2, or 3 substituents that are independently (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylsulfonyl, (C₁-C₆)alkylthio, carboxy, cyano, halogen, halo(C₁-C₆)alkoxy, halo(C₁-C₆)alkyl, hydroxy, hydroxy(C₁-C₆)alkyl, mercapto, nitro, —NR_{M}R_{N}, (NR_{M}R_{N})carbonyl, or oxo;
R_{J} and R_{K} are independently H or (C₁-C₆)alkyl; and
R_{M} and R_{N} are independently H, (C₁-C₆)alkyl, or (C₁-C₆)alkylcarbonyl; or R_{M} and
R_{N} together with the nitrogen they are attached to form a 3 to 8 membered ring; provided that Formula (I) does not encompass:
5-(4-bromophenyl)-1H-indole-3-carboxamide;
5-(2′,6′-dihydroxy-[1,1′-biphenyl]-4-yl)-1H-indole-3-carboxamide; and
5-(2′,6′-dimethoxy-[1,1]biphenyl]-4-yl)-1H-indole-3-carboxamide.

In some embodiments, the compound of formula (I) is

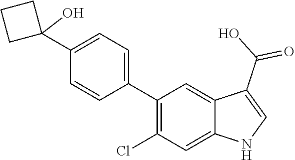 [SMILES: O=C(O)c1c[nH]c2cc(Cl)c(-c3ccc(C4(O)CCC4)cc3)cc12]

The preparation of the compounds of Formula (I), Formula (II) (described hereinbelow), and Compound 1 are described in, for example, WO2014/140704 (also U.S. Pat. No. 8,889,730, issued Nov. 18, 2014, which is incorporated herewith in its entirety).

### BRIEF DESCRIPTION OF THE DRAWINGS

FIGS. 1A-1C show the most expressed AMPK subunit genes quantified in 101,935 single bone marrow cells from eight different donors taken from the Human Cell Atlas (preview.data.humancellatlas.org). The major AMPK isoform in each single cell was estimated based on the gene with the highest expression separately for the α-subunits of AMPK genes (FIG. 1A), β-subunits of AMPK genes (FIG. 1B), and 7-subunits of AMPK genes (FIG. 1C). No isoform was assigned in case of ties for the highest expression or when no expression was detected. The y-axis represents the fraction of cells with a detectable major isoform (averaged across the 8 donors) for 35 bone marrow cell populations defined previously (Hay et al. Exp Hematol, 2018. 68: p. 51-61). PKRAA1 is the name of the gene coding for the α1-AMPK subunit, PKRAA2 for the α2-AMPK subunit, PKRAB1 for the β1-AMPK subunit, PKRAB2 for the β2-AMPK subunit, PKRAG1 for the 71-AMPK subunit, PKRAG2 for the γ2-AMPK subunit and PKRAG3 for the γ3-AMPK subunit.

FIGS. 2A-2C: FIG. 2A shows the result of fetal hemoglobin induction in human CD34+ cells when mobilized CD34+ HSPC from healthy individuals are cultured for 21 days under conditions to promote erythroid differentiation, and are exposed to β1-selective AMPK activator “Compound 1” at the indicated doses (μM). Exposure to AMPK “Compound 1” increases F-cell (HbF+) frequency in enucleated GlyA+ cells in a dose-dependent manner, compared to control (DMSO indicated as Vehicle). FIG. 2B shows the HbF+ fold change to control. (DMSO) cells (A). FIG. 2C: Representative plots from a single donor are shown.

FIG. 3A-3C: FIG. 3A shows the result of “Compound 1” exposure on human CD34+ cells maturation when mobilized CD34+ HSPC from multiple healthy individuals are cultured for 21 days under conditions promoting erythroid. This compound has no impact on enucleation of terminally differentiated erythroid cells compared to control cells (DMSO indicated as Vehicle), based on the frequency of enucleated cells as measured by flow cytometry. FIG. 3B displays that “Compound 1” has no effect on the expression level of erythroid markers CD71 and CD235a during maturation compared to control cells (DMSO indicated as Vehicle). In FIG. 3C representative plots from one healthy donor are shown. All data are expressed as mean±SEM (NS=not significant, n=3).

FIGS. 4A-4B show the result of fetal hemoglobin induction in human CD34+ cells when mobilized CD34+ HSPC from healthy individuals are cultured for 21 days under conditions promoting erythroid differentiation and are exposed to β1-selective AMPK activators “Compound 1” and “Compound 2” at the indicated doses (μM) in FIG. 4A, or to pan-AMPK activators “Compound 3” and “Compound 4” in FIG. 4B. Exposure to “Compound 1” and “Compound 2” increases F-cell (HbF+) frequency in enucleated GlyA+ cells, compared to control cells (DMSO indicated as Vehicle). Similar effect is observed in CD34+ cells exposed to pan-AMPK activators “Compound 3” and “Compound 4,” leading to an increase of F-cell (HbF+) frequency in enucleated GlyA+ cells compared to control cells (DMSO indicated as Vehicle). Representative plots from three healthy donors are shown in FIG. 4A and FIG. 4B, respectively. All data are expressed as mean±SEM (* p<0.05, ** p<0.01, n=3).

FIG. 5A-5C: FIG. 5A shows the result of fetal hemoglobin induction in human CD34+ cells when mobilized CD34+ HSPC from healthy individuals are cultured for 14 days under conditions promoting erythroid differentiation and are exposed to β1-selective AMPK activator “Compound 1” or Hydroxyurea or a combination of both. Exposure to “Compound 1” or Hydroxyurea increases F-cell (HbF+) frequency in enucleated GlyA+ cells, compared to control cells (DMSO indicated as Vehicle). The combination of both leads to a higher frequency of F-cell compared to control and “Compound 1” or HU alone. FIG. 5B shows the HbF+ fold change to control. Representative plots from one healthy donor are shown in FIG. 5C. All data are expressed as mean±SEM (* p<0.05, ** p<0.01, n=3).

FIG. 6A-6C: FIG. 6A shows the result of fetal hemoglobin induction in human CD34+ cells from patients with sickle cell disease after exposure to β1-selective AMPK activators “Compound 1” and “Compound 2”, pan-AMPK activator “Compound 3”, Hydroxyurea, and a combination of “Compound 1” and Hydroxyurea. Exposure to “Compound 1” or Hydroxyurea alone increase F-cell (HbF+) frequency in enucleated GlyA+ cells, compared to control cells (DMSO indicated as Vehicle). The combination of both leads to a higher frequency of F-cell compared to “Compound 1” or Hydroxyurea alone. FIG. 6B shows the HbF+ fold change to control. Representative plots from one donor with sickle cell disease are shown in FIG. 6C. All data are expressed as mean±SEM (n=2).

FIGS. 7A-7E show the expression of pro-inflammation markers CD38 (FIG. 7A), CD64 (FIG. 7B), CD80 (FIG. 7C) and CD86 (FIG. 7D) in M-CSF polarized and activated M1 macrophages from healthy donor. Activation was triggered by LPS (10 ng/mL) or INF-7 (50 ng/mL). Exposure to “Compound 1” prevents the increase of the pro-inflammatory markers induced by LPS and INF-7 compared to control (DMSO indicated as Vehicle). Representative plots for marker CD64 are shown in FIG. 7E. All data are expressed as percentage of total cells (n=1).

FIGS. 8A-8D show the measurement of phosphorylated residue Threonine 172 on the α-domain of AMPK in mobilized CD34+ HSPC from healthy donors at day 11 of erythroid differentiation (FIG. 8A) and in human undifferentiated HUDEP-2 cells (FIG. 8B). Analysis with Alpha SureFire Ultra Multiplex® bead-based assay technology measuring total αAMPK and αAMPK phosphorylated on Thr172 indicates that AMPK phosphorylation peaks at around 3 hour in differentiated CD34+ cells (FIG. 8A) and at around 1 hours in undifferentiated human HUDEP-2 cells (FIG. 8B), when exposed to β1-selective AMPK activators “Compound 1” and “Compound 2”, or to pan-AMPK activators “Compound 3” and “Compound 4”, compared to control DMSO indicated as Vehicle. Representative plots from one healthy donor are shown. All data are expressed as a ratio of signals pAMPK and total AMPK. FIG. 8C shows the measurement of phosphorylated residue Serine 413 on FOXO3 in undifferentiated human HUDEP-2 cells, assessed by Western blotting and using cell lysates from the 1-hour time point. The blot shows that the phosphorylation of FOXO3 on Ser413 increases in the cells exposed to AMPK activators by between 2.5-fold to 5.5-fold based upon quantification of FOXO3 and phospho-FOXO3 (Ser413) expression, normalized to β-actin and assessed by densitometry (FIG. 8D).

FIGS. 9A-9E show in vivo target engagement as measured by increases in α-AMPK phosphorylation, FOXO3 phosphorylation and 7-globin mRNA in bone marrow cells from HbSS Townes SCD mice given “Compound 1” (100 mg/kg, PO) for 2 days. Bone marrow cells were collected 2 hours after the last dose. FIG. 9A displays the analysis with Alpha SureFire Ultra Multiplex® technology measuring total α-AMPK and α-AMPK phosphorylated on Thr172, which shows increased phosphorylation of AMPK (1.5- to 5.5-fold) in the bone marrow of HbAA and HbSS mice given “Compound 1” compared to Vehicle (*p<0.05, n=4 except HbAA mice given “Compound 1” with n=3 or 4). FIG. 9B displays an analysis of the pooled bone marrow lysates by Western blotting and shows that the phosphorylation of FOXO3 on Ser413 increases in the HbAA and HbSS mice given “Compound 1” by between 10- to 12-fold assessed by densitometry (n=1). Graph FIG. 9C indicates fold change to Vehicle-HbAA assessed by quantification of the immunoblot of FIG. 9B. FIG. 9D displays the analysis of mRNA by qPCR, isolated from the bone marrow cells, which shows that the mRNA for the 7-globin gene (HBG) increases around 1.5 fold in the HbSS mice given “Compound 1” (* p<0.05, n=4) but not in HbAA mice after 2 days of oral exposure to “Compound 1”. FIG. 9E displays the measurement of fetal hemoglobin protein in bone marrow cells from HbSS by flowcytometry, which shows an increase of HbF in bone marrow after dosing HbSS mice for 15 days with “Compound 1” (100 mg/kg, PO). (* p<0.05, n=8 to 9).

FIGS. 10A-10B: FIG. 10A shows the effect of AMPK activation by “Compound 1” on Reactive Oxidative Species (ROS) level in bone marrow isolated from HbSS Townes SCD mice given “Compound 1” (100 mg/kg, PO) for 15 days. Measurement of ROS levels by flowcytometry shows a reduction in bone marrow cells from HbSS mice treated with “Compound 1”, compared to control (Vehicle-HbAA) (* p<0.05, n=6 or 7) as displayed in FIG. 10B.

FIGS. 11A-11B: FIG. 11A shows the regulation of markers in human CD34+ cells when mobilized CD34+ HSPC from multiple healthy individuals are exposed to “Compound 1” and are cultured for 14 days under conditions to promote erythroid differentiation. Integrated analysis of transcriptomic and proteomic differential data and correlation of both data set, obtained from CD34+ treated with “Compound 1” for 14 days, shows an upregulation of Heme oxygenase protein (HO-1), a key factor in decreasing oxidative stress response. Ingenuity Pathway Analysis (IPA) of proteomics differential data reveals Nrf2-activated oxidative stress pathway is activated when CD34+ cells are exposed to “Compound 1,” which is displayed in FIG. 11B. Every analysis has been performed on CD34+ cells from three healthy donors.

FIGS. 12A-12C show the effects of indicated doses of “Compound 1” (mg/kg/day), administered for 7 days in a study in rats (QD, PO), on erythrocytes (FIG. 12A), hemoglobin (FIG. 12B) and hematocrit (FIG. 12C). All data are expressed as mean±SD (NS=not significant, n=8).

### DETAILED DESCRIPTION

Although specific embodiments of the present disclosure will now be described with reference to the preparations and schemes, it should be understood that such embodiments are by way of example only and merely illustrative of but a small number of the many possible specific embodiments which can represent applications of the principles of the present disclosure. Various changes and modifications will be obvious to those of skill in the art given the benefit of the present disclosure and are deemed to be within the spirit and scope of the present disclosure as further defined in the appended claims.

The following abbreviations are used herein:
AMPK 5′-adenosine monophosphate-activated protein kinase
AMP adenosine monophosphate
ATP adenosine triphosphate
DMSO dimethyl sulfoxide
GlyA+ cell cell expressing glycophorin A (GYPA, CD235a) by flow cytometry
HBB gene gene expressing the β-globin subunit of adult hemoglobin
HbF fetal hemoglobin
HbS hemoglobin S
HSC hematopoietic stem cells
HSPC hematopoietic stem/progenitor cells
HUDEP-2 Human Umbilical Cord Blood-Derived Erythroid Progenitor-2
INF-γ Interferon gamma
FOXO3 forkhead box 0-3
LPS Lipopolysaccharides
MCV value mean corpuscular volume (a measure of average red blood cell volume)
PO per os (orally)
QD quaque die (once a day)
RBC red blood cells
ROS reactive oxidative species
SCD sickle cell disease
SD standard deviation
SEM standard error of the mean

### Definitions

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present invention, exemplary methods, devices, and materials are now described. All technical and patent publications cited herein are incorporated herein by reference in their entirety. Nothing herein is to be construed as an admission that the invention is not entitled to antedate such disclosure by virtue of prior invention.

The practice of the present disclosure will employ, unless otherwise indicated, conventional techniques of tissue culture, immunology, molecular biology, microbiology, cell biology and recombinant DNA, which are within the skill of the art.

All numerical designations, e.g., pH, temperature, time, concentration, molecular weight, including ranges, are approximations which are varied (+) or (−) by increments of 0.1 or 1.0, where appropriate. It is to be understood, although not always explicitly stated, that all numerical designations are preceded by the term “about”. It also is to be understood, although not always explicitly stated, that the reagents described herein are merely exemplary and that equivalents of such are known in the art.

As used herein, the term “optionally substituted” is meant to be equivalent to the phrase “non-substituted or substituted by.”

As used herein, Compound 2 is a β1-selective AMPK activator o the formula:

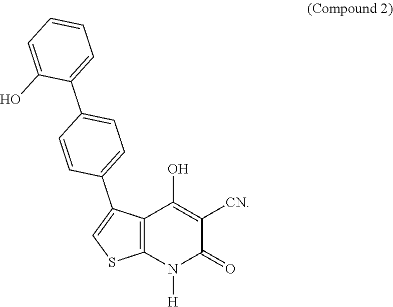 [SMILES: N#Cc1c(O)c2c(-c3ccc(-c4ccccc4O)cc3)csc2[nH]c1=O]

Compound 2 is also known as A-769662 and is available from Sigma-Aldrich (Product Number SML2578). Compound 2 and methods of making the same are described in US2005/0038068, published Feb. 17, 2005.

As used herein, Compound 3 is a pan-selective AMPK activator of the formula:

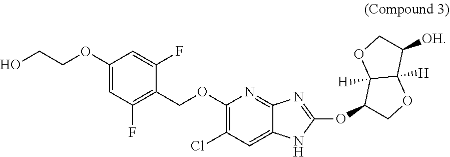 [SMILES: OCCOc1cc(F)c(COc2nc3nc(O[C@@H]4CO[C@H]5[C@@H]4OC[C@H]5O)[nH]c3cc2Cl)c(F)c1]

Compound 3 and methods of making the same are described in WO2014/175330, published Oct. 30, 2014.

As used herein, Compound 4 is a pan-selective AMPK of the formula:

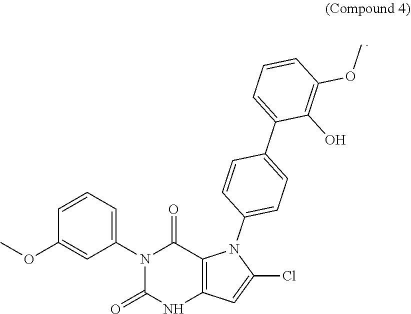

Compound 4 is also known as GSK621 and is available from Sigma-Aldrich (Product Number SML2003). Compound 4 and methods of making the same are described in WO2011/138307, published Nov. 10, 2011.

As used herein, the phrase “in a method of treating or preventing” (such as in the phrase “in a method of treating or preventing a β-hemoglobinopathy”) is meant to be equivalent to the phrase “in the treatment or prevention of” (such as in the phrase “in the treatment or prevention of a β-hemoglobinopathy”).

As used in the specification and claims, the singular form “a”, “an” and “the” include plural references unless the context clearly dictates otherwise. For example, the term “a cell” includes a plurality of cells, including mixtures thereof. Unless specifically stated or obvious from context, as used herein, the term “or” is understood to be inclusive. The term “including” is used herein to mean, and is used interchangeably with, the phrase “including but not limited to”.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transition terms are within the scope of this invention. Use of the term “comprising” herein is intended to encompass “consisting essentially of” and “consisting of”.

A “subject,” “individual” or “patient” is used interchangeably herein, and refers to a vertebrate, such as a mammal. Mammals include, but are not limited to, murines, rats, rabbit, simians, bovines, ovine, porcine, canines, felines, farm animals, sport animals, pets, equines, primates, and humans. In one embodiment, the mammals include horses, dogs, and cats. In some embodiments, the mammal is a human, e.g., a human suffering from a particular disease or disorder, such as Sickle Cell Disease (SCD).

“Administering” is defined herein as a means of providing an agent or a composition containing the agent to a subject in a manner that results in the agent being inside the subject's body. Such an administration can be by any route including, without limitation, oral, transdermal (e.g. vagina, rectum, oral mucosa), by injection (e.g. subcutaneous, intravenous, parenterally, intraperitoneally, into the CNS), or by inhalation (e.g. oral or nasal). Pharmaceutical preparations are, of course, given by forms suitable for each administration route.

“Treating” or “treatment” of a disease generally includes: (1) inhibiting the disease, i.e. arresting or reducing the development of the disease or its clinical symptoms; and/or (2) relieving the disease, i.e. causing regression of the disease or its clinical symptoms.

“Preventing” or “prevention” of a disease generally includes causing the clinical symptoms of the disease not to develop in a patient that may be predisposed to the disease but does not yet experience or display symptoms of the disease.

An “effective amount” or “therapeutically effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, and the route of administration. It is understood, however, that specific dose levels of the therapeutic agents of the present invention for any particular subject depends upon a variety of factors including, for example, the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. In general, one will desire to administer an amount of the compound that is effective to achieve a serum level commensurate with the concentrations found to be effective in vitro. Determination of these parameters is well within the skill of the art. These considerations, as well as effective formulations and administration procedures are well known in the art and are described in standard textbooks. Consistent with this definition, as used herein, the term “therapeutically effective amount” is an amount sufficient to treat (e.g. improve) one or more symptoms associated with a disease or disorder described herein.

As used herein, the term “pharmaceutically acceptable excipient” encompasses any of the standard pharmaceutical excipients, including carriers such as a phosphate buffered saline solution, water, and emulsions, such as an oil/water or water/oil emulsion, and various types of wetting agents. Pharmaceutical compositions also can include stabilizers and preservatives. For examples of carriers, stabilizers and adjuvants, see Remington's Pharmaceutical Sciences (20th ed., Mack Publishing Co. 2000).

As used herein, the term “pharmaceutically acceptable salt” means a pharmaceutically acceptable acid addition salt or a pharmaceutically acceptable base addition salt of a currently disclosed compound that may be administered without any resultant substantial undesirable biological effect(s) or any resultant deleterious interaction(s) with any other component of a

As used herein, the wording “a compound for use . . . ”, for example, shall be understood as being equivalent to the wording “use of a compound for . . . ” or “use of a compound for the preparation of a medicament for use in . . . ”.

The term “(C₂-C₈)alkenylene” means a divalent group derived from a straight or branched chain hydrocarbon of from 2 to 8 carbon atoms containing at least one double bond. Representative examples of alkenylene include, but are not limited to, —CH═CH—, —CH═CH₂CH₂—, and —CH═C(CH₃)CH₂—.

The term “(C₁-C₆)alkoxy” as used herein, means a (C₁-C₆)alkyl group, as defined herein, appended to the parent molecular moiety through an oxygen atom. Representative examples of (C₁-C₆)alkoxy include, but are not limited to, methoxy, ethoxy, propoxy, 2-propoxy, butoxy, tert-butoxy, pentyloxy, and hexyloxy.

The term “(C₁-C₆)alkoxy(C₁-C₆)alkoxy” as used herein, means a (C₁-C₆)alkoxy group, as defined herein, appended to the parent molecular moiety through another (C₁-C₆)alkoxy group, as defined herein. Representative examples of (C₁-C₆)alkoxy(C₁-C₆)alkoxy include, but are not limited to, tert-butoxymethoxy, 2-ethoxyethoxy, 2-methoxyethoxy, and methoxymethoxy.

The term “(C₁-C₆)alkoxy(C₁-C₆)alkyl” as used herein, means a (C₁-C₆)alkoxy group, as defined herein, appended to the parent molecular moiety through a (C₁-C₆))alkyl group, as defined herein. Representative examples of (C₁-C₆)alkoxy(C₁-C₆)alkyl include, but are not limited to, tert-butoxymethyl, 2-ethoxyethyl, 2-methoxyethyl, and methoxymethyl.

The term “(C₁-C₆)alkoxycarbonyl” as used herein, means a (C₁-C₆))alkoxy group, as defined herein, appended to the parent molecular moiety through a carbonyl group, as defined herein. Representative examples of (C₁-C₆)alkoxycarbonyl include, but are not limited to, methoxycarbonyl, ethoxycarbonyl, and tert-butoxycarbonyl.

The term “(C₁-C₆))alkoxysulfonyl” as used herein, means a (C₁-C₆)alkoxy group, as defined herein, appended to the parent molecular moiety through a sulfonyl group, as defined herein. Representative examples of (C₁-C₆)alkoxysulfonyl include, but are not limited to, methoxysulfonyl, ethoxysulfonyl and propoxysulfonyl.

The term “(C₁-C₆)alkyl” as used herein, means a straight or branched chain hydrocarbon containing from 1 to 6 carbon atoms. Representative examples of (C₁-C₆)alkyl include, but are not limited to, methyl, ethyl, n-propyl, iso-propyl, n-butyl, sec-butyl, iso-butyl, tert-butyl, n-pentyl, isopentyl, neopentyl, and n-hexyl.

The term “(C₁-C₆)alkylcarbonyl” as used herein, means a (C₁-C₆)alkyl group, as defined herein, appended to the parent molecular moiety through a carbonyl group, as defined herein. Representative examples of (C₁-C₆))alkylcarbonyl include, but are not limited to, acetyl, 1-oxopropyl, 2,2-dimethyl-1-oxopropyl, 1-oxobutyl, and 1-oxopentyl.

The term “(C₁-C₆)alkylene” means a divalent group derived from a straight or branched chain hydrocarbon of from 1 to 6 carbon atoms. Representative examples of (C₁-C₆)alkylene include, but are not limited to, —CH₂—, —CH(CH₃)—, —C(CH₃)₂—, —CH₂CH₂—, —CH₂CH₂CH₂—, —CH₂CH₂CH₂CH₂—, —CH₂CH(CH₃)CH₂—, and —CH₂CH₂CH₂CH₂CH₂CH₂—.

The term “(C₁-C₆)alkylsulfonyl” as used herein, means an (C₁-C₆)alkyl group, as defined herein, appended to the parent molecular moiety through a sulfonyl group, as defined herein. Representative examples of (C₁-C₆)alkylsulfonyl include, but are not limited to, methylsulfonyl and ethylsulfonyl.

The term “(C₁-C₆)alkylthio” as used herein, means a (C₁-C₆)alkyl group, as defined herein, appended to the parent molecular moiety through a sulfur atom. Representative examples of (C₁-C₆)alkylthio include, but are not limited to, methylthio, ethylthio, tert-butylthio, and hexylthio.

The term “aryl” as used herein, means a phenyl or naphthyl group.

The term “aryl(C₁-C₆)alkoxy” as used herein, means an aryl group, as defined herein, appended to the parent molecular moiety through an (C₁-C₆)alkoxy group, as defined herein.

The term “aryl(C₁-C₆)alkyl” as used herein, means an aryl group, as defined herein, appended to the parent molecular moiety through an (C₁-C₆)alkyl group, as defined herein. Representative examples of aryl(C₁-C₆)alkyl include, but are not limited to, benzyl, 2-phenylethyl, 3-phenylpropyl, and 2-naphth-2-ylethyl.

The term “arylcarbonyl” as used herein, means an aryl group, as defined herein, appended to the parent molecular moiety through a carbonyl group, as defined herein. Examples of arylcarbonyl are benzoyl and naphthoyl.

The term “aryloxy” as used herein, means an aryl group, as defined herein, appended to the parent molecular moiety through an oxygen atom. Examples of aryloxy are phenoxy and naphthalenyloxy.

The term “carbonyl” as used herein, means a —C(O)— group.

The term “carboxy” as used herein, means a —C(O)OH group.

The term “carboxy(C₁-C₆)alkoxy” as used herein, means a carboxy group, as defined herein, is attached to the parent molecular moiety through a (C₁-C₆)alkoxy group, as defined herein.

The term “carboxy(C₁-C₆)alkyl” as used herein, means a carboxy group, as defined herein, is attached to the parent molecular moiety through a (C₁-C₆)alkyl group, as defined herein.

The term “cyano” as used herein, means a —CN group.

The term “(C₃-C₈)cycloalkyl” as used herein, means a saturated cyclic hydrocarbon group containing from 3 to 8 carbons, examples of (C₃-C₈)cycloalkyl include cyclopropyl, cyclobutyl, cyclopentyl, cyclohexyl, cycloheptyl, and cyclooctyl.

The term “(C₃-C₈)cycloalkyl(C₁-C₆)alkoxy” as used herein, means a (C₃-C₈)cycloalkyl group, as defined herein, appended to the parent molecular moiety through a (C₁-C₆)alkoxy group, as defined herein.

The term “(C₃-C₈)cycloalkyl(C₁-C₆)alkyl” as used herein, means a (C₃-C₈)cycloalkyl group, as defined herein, appended to the parent molecular moiety through a (C₁-C₆)alkyl group, as defined herein. Representative examples of (C₃-C₈)cycloalkyl(C₁-C₆)alkyl include, but are not limited to, cyclopropylmethyl, 2-cyclobutylethyl, cyclopentylmethyl, cyclohexylmethyl, and 4-cycloheptylbutyl.

The term “(C₃-C₈)cycloalkylcarbonyl” as used herein, means (C₃-C₈)cycloalkyl group, as defined herein, appended to the parent molecular moiety through a carbonyl group, as defined herein. Representative examples of (C₃-C₈)cycloalkylcarbonyl include, but are not limited to, cyclopropylcarbonyl, 2-cyclobutylcarbonyl, and cyclohexylcarbonyl.

The term “(C₃-C₈)cycloalkyloxy” as used herein, means (C₃-C₈)cycloalkyl group, as defined herein, appended to the parent molecular moiety through an oxygen atom, as defined herein. Representative examples of (C.sub.3-C.sub.8)cycloalkyloxy include, but are not limited to, cyclopropyloxy, cyclobutyloxy, cyclopentyloxy, cyclohexyloxy, cycloheptyloxy, and cyclooctyloxy.

The term “halo” or “halogen” as used herein, means —Cl, —Br, —I or —F.

The term “halo(C₁-C₆)alkoxy” as used herein, means at least one halogen, as defined herein, appended to the parent molecular moiety through a (C₁-C₆)alkoxy group, as defined herein. Representative examples of halo(C₁-C₆)alkoxy include, but are not limited to, chloromethoxy, 2-fluoroethoxy, trifluoromethoxy, and pentafluoroethoxy.

The term “halo(C₁-C₆)alkyl” as used herein, means at least one halogen, as defined herein, appended to the parent molecular moiety through a (C₁-C₆)alkyl group, as defined herein. Representative examples of halo(C₁-C₆)alkyl include, but are not limited to, chloromethyl, 2-fluoroethyl, trifluoromethyl, pentafluoroethyl, and 2-chloro-3-fluoropentyl.

The term “heteroaryl,” as used herein, means a monocyclic heteroaryl or a bicyclic heteroaryl. The monocyclic heteroaryl is a 5 or 6 membered ring. The 5 membered ring consists of two double bonds and one, two, three or four nitrogen atoms and/or optionally one oxygen or sulfur atom. The 6 membered ring consists of three double bonds and one, two, three or four nitrogen atoms. The 5 or 6 membered heteroaryl is connected to the parent molecular moiety through any carbon atom or any nitrogen atom contained within the heteroaryl. Representative examples of monocyclic heteroaryl include, but are not limited to, furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, and triazinyl. The bicyclic heteroaryl consists of a monocyclic heteroaryl fused to a phenyl, or a monocyclic heteroaryl fused to a cycloalkyl, or a monocyclic heteroaryl fused to a cycloalkenyl, or a monocyclic heteroaryl fused to a monocyclic heteroaryl. The bicyclic heteroaryl is connected to the parent molecular moiety through any carbon atom or any nitrogen atom contained within the bicyclic heteroaryl. Representative examples of bicyclic heteroaryl include, but are not limited to, benzimidazolyl, benzofuranyl, benzothienyl, benzoxadiazolyl, cinnolinyl, dihydroquinolinyl, dihydroisoquinolinyl, furopyridinyl, indazolyl, indolyl, isoquinolinyl, naphthyridinyl, quinolinyl, tetrahydroquinolinyl, and thienopyridinyl.

The term “heteroaryl(C₁-C₆)alkoxy” as used herein, means a heteroaryl group, as defined herein, appended to the parent molecular moiety through an (C₁-C₆))alkoxy group, as defined herein. Representative examples of heteroaryl(C₁-C₆)alkoxy include, but are not limited to, fur-3-ylmethoxy, 1H-imidazol-2-ylmethoxy, 1H-imidazol-4-ylmethoxy, 1-(pyridin-4-yl)ethoxy, pyridin-3-ylmethoxy, 6-chloropyridin-3-ylmethoxy, pyridin-4-ylmethoxy, (6-(trifluoromethyl)pyridin-3-yl)methoxy, (6-(cyano)pyridin-3-yl)methoxy, (2-(cyano)pyridin-4-yl)methoxy, (5-(cyano)pyridin-2-yl)methoxy, (2-(chloro)pyridin-4-yl)methoxy, pyrimidin-5-ylmethoxy, 2-(pyrimidin-2-yl)propoxy, thien-2-ylmethoxy, and thien-3-ylmethoxy.

The term “heteroaryl(C₁-C₆)alkyl” as used herein, means a heteroaryl, as defined herein, appended to the parent molecular moiety through an (C₁-C₆)alkyl group, as defined herein. Representative examples of heteroaryl(C₁-C₆)alkyl include, but are not limited to, fur-3-ylmethyl, 1H-imidazol-2-ylmethyl, 1H-imidazol-4-ylmethyl, 1-(pyridin-4-yl)ethyl, pyridin-3-ylmethyl, 6-chloropyridin-3-ylmethyl, pyridin-4-ylmethyl, (6-(trifluoromethyl)pyridin-3-yl)methyl, (6-(cyano)pyridin-3-yl)methyl, (2-(cyano)pyridin-4-yl)methyl, (5-(cyano)pyridin-2-yl)methyl, (2-(chloro)pyridin-4-yl)methyl, pyrimidin-5-ylmethyl, 2-(pyrimidin-2-yl)propyl, thien-2-ylmethyl, and thien-3-ylmethyl.

The term “heteroarylcarbonyl” as used herein, means a heteroaryl group, as defined herein, appended to the parent molecular moiety through a carbonyl group, as defined herein. Representative examples of heteroarylcarbonyl include, but are not limited to, fur-3-ylcarbonyl, 1H-imidazol-2-ylcarbonyl, 1H-imidazol-4-ylcarbonyl, pyridin-3-ylcarbonyl, 6-chloropyridin-3-ylcarbonyl, pyridin-4-ylcarbonyl, (6-(trifluoromethyl)pyridin-3-yl)carbonyl, (6-(cyano)pyridin-3-yl)carbonyl, (2-(cyano)pyridin-4-yl)carbonyl, (5-(cyano)pyridin-2-yl)carbonyl, (2-(chloro)pyridin-4-yl)carbonyl, pyrimidin-5-ylcarbonyl, pyrimidin-2-ylcarbonyl, thien-2-ylcarbonyl, and thien-3-ylcarbonyl.

The term “heteroaryloxy” as used herein, means a heteroaryl group, as defined herein, appended to the parent molecular moiety through an oxygen atom. Representative examples of heteroaryloxy include, but are not limited to, fur-3-yloxy, 1H-imidazol-2-yloxy, 1H-imidazol-4-yloxy, pyridin-3-yloxy, 6-chloropyridin-3-yloxy, pyridin-4-yloxy, (6-(trifluoromethyl)pyridin-3-yl)oxy, (6-(cyano)pyridin-3-yl)oxy, (2-(cyano)pyridin-4-yl)oxy, (5-(cyano)pyridin-2-yl)oxy, (2-(chloro)pyridin-4-yl)oxy, pyrimidin-5-yloxy, pyrimidin-2-yloxy, thien-2-yloxy, and thien-3-yloxy.

The term “(C₃-C₇)heterocycle” or “(C₃-C₇)heterocyclic” as used herein, means a 3, 4, 5, 6 or 7 membered ring containing at least one heteroatom independently selected from the group consisting of 0, N, and S. The 3 or 4 membered ring contains 1 heteroatom selected from the group consisting of O, N and S. The 5 membered ring contains zero or one double bond and one, two or three heteroatoms selected from the group consisting of O, N and S. The 6 or 7 membered ring contains zero, one or two double bonds and one, two or three heteroatoms selected from the group consisting of O, N and S. The heterocycle is connected to the parent molecular moiety through any carbon atom or any nitrogen atom contained within the heterocycle. Representative examples of heterocycle include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl.

The term “(C₃-C₇)heterocycle(C.sub.1-C.sub.6)alkoxy” as used herein, means a 3-7 membered heterocycle group, as defined herein, appended to the parent molecular moiety through an (C.sub.1-C.sub.6)alkoxy group, as defined herein.

The term “(C₃-C₇)heterocycle(C₁-C₆)alkyl” as used herein, means a 3-7 membered heterocycle, as defined herein, appended to the parent molecular moiety through an (C.sub.1-C.sub.6)alkyl group, as defined herein.

The term “(C₃-C₇)heterocyclecarbonyl” as used herein, means a 3-7 membered heterocycle, as defined herein, appended to the parent molecular moiety through a carbonyl group, as defined herein.

The term “(C₃-C₇)heterocycleoxy” as used herein, means a 3-7 membered heterocycle, as defined herein, appended to the parent molecular moiety through an oxygen atom.

The term “hydroxy” as used herein, means an —OH group.

The term “hydroxy(C₁-C₆)alkoxy” as used herein, means at least one hydroxy group, as defined herein, is appended to the parent molecular moiety through a (C₁-C₆)alkoxy group, as defined herein. Representative examples of hydroxy(C₁-C₆)alkoxy include, but are not limited to, hydroxymethoxy, 2-hydroxyethoxy, 3-hydroxypropoxy, 2,3-dihydroxypentoxy, and 2-ethyl-4-hydroxyheptoxy.

The term “hydroxy(C₁-C₆)alkyl” as used herein, means at least one hydroxy group, as defined herein, is appended to the parent molecular moiety through a (C₁-C₆)alkyl group, as defined herein. Representative examples of hydroxy(C₁-C₆)alkyl include, but are not limited to, hydroxymethyl, 2-hydroxyethyl, 3-hydroxypropyl, 2,3-dihydroxypentyl, and 2-ethyl-4-hydroxyheptyl.

The term “mercapto” as used herein, means a —SH group.

The term “nitro” as used herein, means a —NO₂ group.

The term “NR_{E}R_{F}” as used herein, means two groups, R_{E} and R_{F}, which are appended to the parent molecular moiety through a nitrogen atom. R_{E} and R_{F} are each independently H or (C₁-C₆)alkyl. Representative examples of NR.sub.ER.sub.F include, but are not limited to, amino, methylamino, dimethylamino, and ethylmethylamino.

The term “(NR_{E}R_{F})carbonyl” as used herein, means a NR_{E}R_{F} group, as defined herein, appended to the parent molecular moiety through a carbonyl group, as defined herein. Representative examples of (NR_{E}R_{F})carbonyl include, but are not limited to, aminocarbonyl, (methylamino)carbonyl, (dimethylamino)carbonyl, and (ethylmethylamino)carbonyl.

The term “NR_{G}R_{H}” as used herein, means two groups, R_{G} and R_{H}, which are appended to the parent molecular moiety through a nitrogen atom. R_{G} and R_{H} are each independently H, (C₁-C₆))alkyl, or (C₁-C₆)alkylcarbonyl. Representative examples of NR_{G}R_{H} include, but are not limited to, amino, methylamino, dimethylamino, ethylmethylamino, acetamido, propionamido, and isobutyramido.

The term “(NR_{G}R_{H})carbonyl” as used herein, means a NR_{G}R_{H} group, as defined herein, appended to the parent molecular moiety through a carbonyl group, as defined herein. Representative examples of (NR_{G}R_{H})carbonyl include, but are not limited to, aminocarbonyl, (methylamino)carbonyl, (dimethylamino)carbonyl, and (ethylmethylamino)carbonyl.

The term “NR_{J}R_{K}” as used herein, means two groups, R_{J} and R_{K}, which are appended to the parent molecular moiety through a nitrogen atom. R_{J} and R_{K} are each independently H or (C₁-C₆)alkyl. Representative examples of R_{J} and R_{K} include, but are not limited to, amino, methylamino, dimethylamino, and ethylmethylamino.

The term “(NR_{J}R_{K})carbonyl” as used herein, means a NR_{J}R_{K} group, as defined herein, appended to the parent molecular moiety through a carbonyl group, as defined herein. Representative examples of (NR_{J}R_{K})carbonyl include, but are not limited to, aminocarbonyl, (methylamino)carbonyl, (dimethylamino)carbonyl, and (ethylmethylamino)carbonyl.

The term “NR_{M}R_{N}” as used herein, means two groups, R_{M} and R_{N}, which are appended to the parent molecular moiety through a nitrogen atom. R_{M} and R_{N} are each independently H, (C₁-C₆)alkyl, or (C₁-C₆)alkylcarbonyl; or R_{M} and R_{N} together with the nitrogen they are attached to form a 3 to 8 membered ring. Representative examples of R_{M} and R_{N} include, but are not limited to, amino, methylamino, dimethylamino, ethylmethylamino, aziridinyl, azetidinyl, pyrrolidinyl, piperidinyl, azepanyl, and azocanyl.

The term “NR_{M}R_{N}(C₁-C₆)alkoxy” as used herein, means a NR_{M}R_{N} group, as defined herein, appended to the parent molecular moiety through a (C₁-C₆)alkoxy group, as defined herein.

The term “NR_{M}R_{N}((C₁-C₆)alkyl” as used herein, means a NR_{M}R_{N} group, as defined herein, appended to the parent molecular moiety through a (C₁-C₆)alkyl group, as defined herein.

The term “(NR_{M}R_{N})carbonyl” as used herein, means a NR_{M}R_{N} group, as defined herein, appended to the parent molecular moiety through a carbonyl group, as defined herein. Representative examples of (NR_{M}R_{N})carbonyl include, but are not limited to, aminocarbonyl, (methylamino)carbonyl, (dimethylamino)carbonyl, and (ethylmethylamino)carbonyl.

The term “(NR_{M}R_{N})carbonyl(C₁-C₆)alkoxy” as used herein, means a (NR_{M}R_{N})carbonyl group, as defined herein, appended to the parent molecular moiety through a (C₁-C₆)alkoxy group, as defined herein.

The term “(NR_{M}R_{N})carbonyl(C₁-C₆)alkyl” as used herein, means a (NR_{M}R_{N})carbonyl group, as defined herein, appended to the parent molecular moiety through a (C₁-C₆)alkyl group, as defined herein. The recitation of a listing of chemical groups in any definition of a variable herein includes definitions of that variable as any single group or combination of listed groups.

The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### Compounds

The present disclosure relates to methods utilizing a β1-selective AMPK activator for use in therapeutic methods relating to the treatment or prevention of the diseases and disorders discussed herein.

In some aspects, provided herein are methods of utilizing a β1-selective AMPK activator of Formula (I),

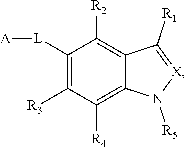

or a pharmaceutically acceptable salt thereof, wherein
X is N or CH;
R₁ is —C(O)OR_{A}, —C(O)NR_{B}R_{C}, —S(O₂)OR_{A}, —S(O₂)NHC(O)R_{D},
5-Oxo-4,5-dihydro-1,2,4-oxadiazol-3-yl, or 1H-tetrazol-5-yl;
R_{A} is H or (C₁-C₆)alkyl;
R_{B} and R_{C} are independently H, (C₁-C₆)alkyl, or —S(O₂)R_{D};
R_{D} is (C₁-C₆)alkyl, —CF₃, or phenyl, wherein the phenyl is optionally substituted with 1, 2, 3, 4, or 5 substituents that are independently (C₁-C₆)alkoxy, (C₁-C₆)alkyl, cyano, halogen, halo(C₁-C₆)alkoxy, halo(C₁-C₆)alkyl, hydroxy, mercapto, nitro, or NR_{E}R_{F};
R_{E} and R_{F} are independently H or (C₁-C₆)alkyl;
R₂, R₃, and R₄ are independently H, (C₁-C₆)alkoxy, (C₁-C₆)alkyl, (C₁-C₆)alkylthio, carboxy, cyano, halogen, halo(C₁-C₆)alkoxy, halo(C₁-C₆)alkyl, hydroxy,hydroxy(C₁-C₈)alkyl, mercapto, nitro, —NR_{G}R_{H} or (NR_{G}R_{H})carbonyl;
R_{G} and R_{H} are independently H, (C₁-C₆)alkyl, or (C₁-C₆)alkylcarbonyl;
R₅ is H or (C₁-C₆)alkyl;
L is a bond, O, S, NRA, (C₁-C₆)alkylene, (C₂-C₆)alkenylene, or (C₂-C₆)alkynylene;
A is phenyl, 2,3-dihydrobenzo[b][1,4]dioxinyl, 2,3-dihydrobenzofuranyl,
2,3-dihydro-1H-indenyl, imidazolyl, pyrazinyl, pyrazolyl, pyridinyl, pyrimidinyl, or thiazolyl, wherein each is optionally substituted with 1, 2, 3, 4, or 5 substituents that are independently (C₁-C₆)alkoxy, (C₁-C₆)alkoxy(C₁-C₆)alkoxy, (C₁-C₆)alkoxy(C₁-C₆)alkyl, (C₁-C₆)alkoxycarbonyl, (C₁-C₆)alkyl, (C₁-C₆)alkylcarbonyl, (C₁-C₆)alkylthio, aryl, aryl(C₁-C₆)alkoxy, aryl(C₁-C₆)alkyl, arylcarbonyl, aryloxy, carboxy,
carboxy(C₁-C₆)alkoxy, carboxy(C₁-C₆)alkyl, cyano, (C₃-C₈)cycloalkyl,
(C₃-C₈)cycloalkyl(C₁-C₆)alkoxy, (C₃-C₈)cycloalkyl(C₁-C₆)alkyl, (C₃-C₈)cycloalkylcarbonyl, (C₃-C₈)cycloalkyloxy, halogen, halo(C₁-C₆)alkoxy, halo(C₁-C₆)alkyl, heteroaryl, heteroaryl(C₁-C₆)alkoxy, heteroaryl(C₁-C₆)alkyl, heteroarylcarbonyl, heteroaryloxy, (C₃-C₇)heterocycle, (C₃-C₇)heterocycle(C₁-C₆)alkoxy, (C₃-C₇)heterocycle(C₁-C₆)alkyl, (C₃-C₇)heterocyclecarbonyl, (C₃-C₇)heterocyclecarbonyl(C₁-C₆)alkyl,
(C₃-C₇)heterocycleoxy, hydroxy, hydroxy(C₁-C₆)alkoxy, hydroxy(C₁-C₆)alkyl, mercapto, nitro, —NR_{J}R_{K}, (NR_{J}R_{K})carbonyl, —NR_{M}R_{N}, —NR_{M}R_{N}(C₁-C₆)alkoxy, (NR_{M}R_{N})carbonyl, (NR_{M}R_{N})carbonyl(C₁-C₆)alkyl, or (NR_{M}R_{N})carbonyl(C₁-C₆)alkoxy; wherein the aryl, aryl(C₁-C₆)alkoxy, aryl(C₁-C₆)alkyl, arylcarbonyl, and aryloxy are optionally substituted with 1, 2, 3, 4, or 5 substituents that are independently (C₁-C₆)alkoxy,
(C₁-C₆)alkoxycarbonyl, (C₁-C₆)alkyl, (C₁-C₆)alkylcarbonyl, (C₁-C₆)alkylthio, carboxy, cyano, halogen, halo(C₁-C₆)alkoxy, halo(C₁-C₆)alkyl, hydroxy, hydroxy(C₁-C₆)alkyl, mercapto, nitro, —NR_{M}R_{N}, or (NR_{M}R_{N})carbonyl; wherein the halo(C₁-C₆)alkyl is optionally substituted with 1 or 2 hydroxy groups; wherein the (C₃-C₈)cycloalkyl,
(C₃-C₈)cycloalkyl(C₁-C₆)alkoxy, (C₃-C₈)cycloalkyl(C₁-C₆)alkyl, (C₃-C₈)cycloalkylcarbonyl, and (C₃-C₈)cycloalkyloxy are optionally substituted with 1, 2, or 3 substituents that are independently (C₁-C₆)alkoxy, (C₁-C₆)alkoxycarbonyl, (C₁-C₆)alkyl, (C₁-C₆)alkylcarbonyl, (C₁-C₆)alkylthio, carboxy, cyano, halogen, halo(C₁-C₆)alkoxy, halo(C₁-C₆)alkyl, hydroxy, hydroxy(C₁-C₆)alkyl, mercapto, nitro, —NR_{M}R_{N}, or (NR_{M}R_{N})carbonyl; wherein the heteroaryl, heteroaryl(C₁-C₆)alkoxy, heteroaryl(C₁-C₆)alkyl, heteroarylcarbonyl, and heteroaryloxy, are optionally substituted with 1, 2, or 3 substituents that are independently (C₁-C₆)alkoxy, (C₁-C₆)alkoxycarbonyl, (C₁-C₆)alkyl, (C₁-C₆)alkylcarbonyl, (C₁-C₆)alkylthio, carboxy, cyano, halogen, halo(C₁-C₆)alkoxy, halo(C₁-C₆)alkyl, hydroxy, hydroxy(C₁-C₆)alkyl, mercapto, nitro, —NR_{M}R_{N}, or (NR_{M}R_{N})carbonyl; and wherein the (C₃-C₇)heterocycle, (C₃-C₇)heterocycle(C₁-C₆)alkoxy, (C₃-C₇)heterocycle(C₁-C₆)alkyl, (C₃-C₇)heterocyclecarbonyl, (C₃-C₇)heterocyclecarbonyl(C₁-C₆)alkyl, and
(C₃-C₇)heterocycleoxy, are optionally substituted with 1, 2, or 3 substituents that are independently (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylsulfonyl, (C₁-C₆)alkylthio, carboxy, cyano, halogen, halo(C₁-C₆)alkoxy, halo(C₁-C₆)alkyl, hydroxy, hydroxy(C₁-C₆)alkyl, mercapto, nitro, —NR_{M}R_{N}, (NR_{M}R_{N})carbonyl, or oxo;
R_{J} and R_{K} are independently H or (C₁-C₆)alkyl; and
R_{M} and R_{N} are independently H, (C₁-C₆)alkyl, or (C₁-C₆)alkylcarbonyl; or R_{M} and
R_{N} together with the nitrogen they are attached to form a 3 to 8 membered ring;
provided that Formula (I) does not encompass:
5-(4-bromophenyl)-1H-indole-3-carboxamide;
5-(2′,6′-dihydroxy-[1,1′-biphenyl]-4-yl)-1H-indole-3-carboxamide; and
5-(2′,6′-dimethoxy-[1,1]biphenyl]-4-yl)-1H-indole-3-carboxamide.

In some embodiments, the β1-selective AMPK activator is a compound of Formula (I)

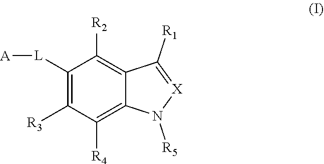

or a pharmaceutically acceptable salt thereof, wherein
X is N or CH;
L is a bond, O, S, NRA, (C₁-C₆)alkylene, (C₂-C₆)alkenylene, or (C₂-C₆)alkynylene;

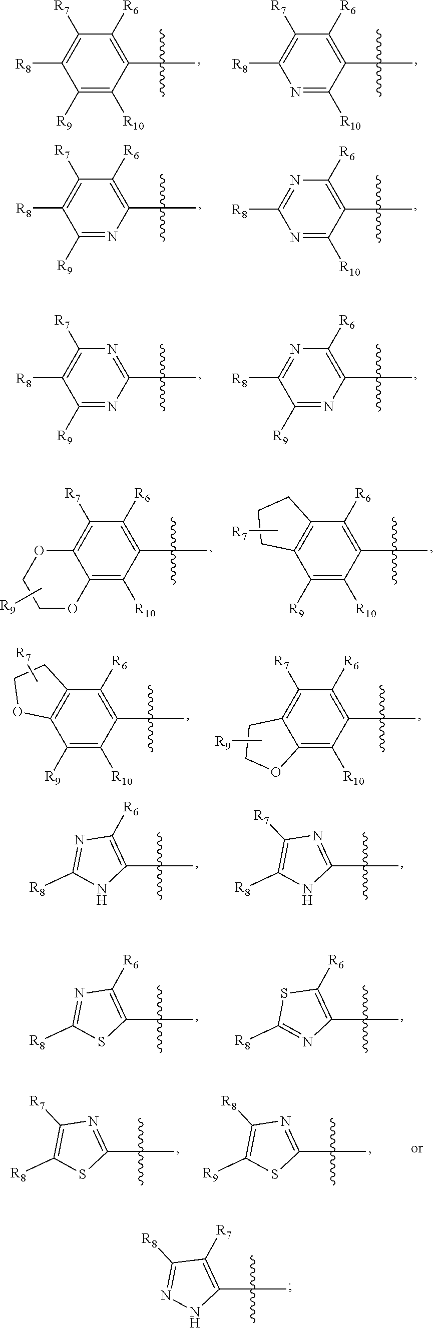

Ri is —C(O)OR_{A}, —C(O)NR_{B}R_{C}, —S(O₂)OR_{A}, —S(O₂)NHC(O)R_{D},
5-oxo-4,5-dihydro-1,2,4-oxadiazol-3-yl, or 1H-tetrazol-5-yl;
R_{A} is H or (C₁-C₆)alkyl;
R_{B} and R_{C} are independently H, (C₁-C₆)alkyl, or —S(O₂)R_{D};
R_{D} is (C₁-C₆)alkyl, —CF₃, or phenyl, wherein the phenyl is optionally substituted with 1, 2, 3, 4, or 5 substituents that are independently (C₁-C₆)alkoxy, (C₁-C₆)alkyl, cyano, halogen, halo(C₁-C₆)alkoxy, halo(C₁-C₆)alkyl, hydroxy, mercapto, nitro, or NR_{E}R_{F};
R_{E} and R_{F} are independently H or (C₁-C₆)alkyl;
R₂, R₃, and R₄ are independently H, (C₁-C₆)alkoxy, (C₁-C₆)alkyl, (C₁-C₆)alkylthio, carboxy, cyano, halogen, halo(C₁-C₆)alkoxy, halo(C₁-C₆)alkyl, hydroxy, hydroxy(C₁-C₈)alkyl, mercapto, nitro, —NR_{G}R_{H}, or (NR_{G}R_{H})carbonyl;
R_{G} and R_{H} are independently H, (C₁-C₆) alkyl, or (C₁-C₆) alkylcarbonyl;
R₅ is H or (C₁-C₆)alkyl;
R₆, R₇, R₉, and R₁₀ are independently H, (C₁-C₆)alkoxy, (C₁-C₆)alkoxycarbonyl, (C₁-C₆)alkyl, (C₁-C₆)alkylcarbonyl, (C₁-C₆)alkylthio, carboxy, cyano, halogen, halo(C₁-C₆)alkoxy, halo(C₁-C₆)alkyl, hydroxy, hydroxy(C₁-C₆)alkyl, mercapto, nitro, —NR_{J}R_{K}, or (NR_{J}R_{K})carbonyl;
R_{J} and R_{K} are independently H or (CrC₆)alkyl;
R₈ is H, (C₁-C₆)alkoxy, (C₁-C₆)alkoxy(C₁-C₆)alkoxy, (C₁-C₆)alkoxy(C₁-C₆)alkyl, (C₁-C₆)alkoxycarbonyl, (C₁-C₆)alkyl, (C₁-C₆)alkylcarbonyl, (C₁-C₆)alkylthio, aryl, aryl(C₁-C₆)alkoxy, aryl(C₁-C₆)alkyl, arylcarbonyl, aryloxy, carboxy, carboxy(C₁-C₆)alkoxy, carboxy(C₁-C₆)alkyl, cyano, (C₃-C₈)cycloalkyl, (C₃-C₈)cycloalkyl(C₁-C₆)alkoxy, (C₃-C₈)cycloalkyl(C₁-C₆)alkyl, (C₃-C₈)cycloalkylcarbonyl, (C₃-C₈)cycloalkyloxy, halogen, halo(C₁-C₆)alkoxy, halo(C₁-C₆)alkyl, heteroaryl, heteroaryl(C₁-C₆)alkoxy, heteroaryl(C₁-C₆)alkyl, heteroarylcarbonyl, heteroaryloxy, (C₃-C₇)heterocycle, (C₃-C₇)heterocycle(C₁-C₆)alkoxy, (C₃-C₇)heterocycle(C₁-C₆)alkyl, (C₃-C₇)heterocyclecarbonyl, (C₃-C₇)heterocyclecarbonyl(C₁-C₆)alkyl, (C₃-C₇)heterocycleoxy, hydroxy, hydroxy(C₁-C₆)alkoxy, hydroxy(C₁-C₆)alkyl, mercapto, nitro, —NR_{M}R_{N}, —NR_{M}R_{N}(C₁-C₆)alkoxy, (NR_{M}R_{N})carbonyl, (NR_{M}R_{N})carbonyl(C₁-C₆)alkyl, or
(NR_{M}R_{N})carbonyl(C₁-C₆)alkoxy; wherein the aryl, aryl(C₁-C₆)alkoxy, aryl(C₁-C₆)alkyl, arylcarbonyl, and aryloxy are optionally substituted with 1, 2, 3, 4, or 5 substituents that are independently (C₁-C₆)alkoxy, (C₁-C₆)alkoxycarbonyl, (C₁-C₆)alkyl, (C₁-C₆)alkylcarbonyl, (C₁-C₆)alkylthio, carboxy, cyano, halogen, halo(C₁-C₆)alkoxy, halo(C₁-C₆)alkyl, hydroxy, hydroxy(C₁-C₆)alkyl, mercapto, nitro, —NR_{M}R_{N}, or
(NR_{M}R_{N})carbonyl; wherein the halo(C₁-C₆)alkyl is optionally substituted with 1 or 2 hydroxy groups; wherein the (C₃-C₈)cycloalkyl, (C₃-C₈)cycloalkyl(C₁-C₆)alkoxy, (C₃-C₈)cycloalkyl(C₁-C₆)alkyl, (C₃-C₈)cycloalkylcarbonyl, and (C₃-C₈)cycloalkyloxy are optionally substituted with 1, 2, or 3 substituents that are independently (C₁-C₆)alkoxy, (C₁-C₆)alkoxycarbonyl, (C₁-C₆)alkyl, (C₁-C₆)alkylcarbonyl, (C₁-C₆)alkylthio, carboxy, cyano, halogen, halo(C₁-C₆)alkoxy, halo(C₁-C₆)alkyl, hydroxy, hydroxy(C₁-C₆)alkyl, mercapto, nitro, —NR_{M}R_{N}, or (NR_{M}R_{N})carbonyl; wherein the heteroaryl, heteroaryl(C₁-C₆)alkoxy, heteroaryl(C₁-C₆)alkyl, heteroarylcarbonyl, and heteroaryloxy, are optionally substituted with 1, 2, or 3 substituents that are independently (d-C₆)alkoxy, (d-C₆)alkoxycarbonyl, (C₁-C₆)alkyl, (C₁-C₆)alkylcarbonyl, (C₁-C₆)alkylthio, carboxy, cyano, halogen, halo(C₁-C₆)alkoxy, halo(C₁-C₆)alkyl, hydroxy, hydroxy(C₁-C₆)alkyl, mercapto, nitro, —NR_{M}R_{N}, or (NR_{M}R_{N})carbonyl; and wherein the (C₃-C₇)heterocycle, (C₃-C₇)heterocycle(C₁-C₆)alkoxy, (C₃-C₇)heterocycle(C₁-C₆)alkyl, (C₃-C₇)heterocyclecarbonyl, (C₃-C₇)heterocyclecarbonyl(C₁-C₆)alkyl, and
(C₃-C₇)heterocycleoxy, are optionally substituted with 1, 2, or 3 substituents that are independently (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylsulfonyl, (C₁-C₆)alkylthio, carboxy, cyano, halogen, halo(C₁-C₆)alkoxy, halo(C₁-C₆)alkyl, hydroxy, hydroxy(C₁-C₆)alkyl, mercapto, nitro, —NR_{M}R_{N}, (NR_{M}R_{N})carbonyl, or oxo; and
R_{M} and R_{N} are independently H, (C₁-C₆)alkyl, or (C₁-C₆)alkylcarbonyl; or R_{M} and R_{N} together with the nitrogen they are attached to form a 3 to 8 membered ring;
provided that Formula (I) does not encompass:
5-(4-bromophenyl)-1H-indole-3-carboxamide;
5-(2′,6′-dihydroxy-[1,1′-biphenyl]-4-yl)-1H-indole-3-carboxamide; and
5-(2′,6′-dimethoxy-[1,1]biphenyl]-4-yl)-1H-indole-3-carboxamide.

In another embodiment, the β1-selective AMPK activator is a compound of Formula (I), or a pharmaceutically acceptable salt thereof, wherein X is N or CH; L is a bond or —C₆)alkynylene;
A is

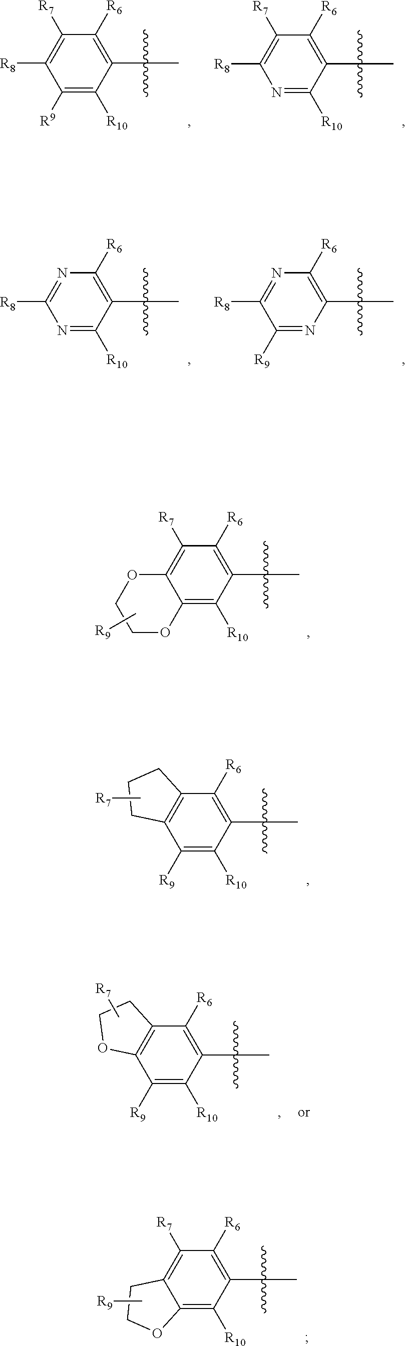

R₁ is —C(O)OR_{A}, —C(O)R_{B}R_{C}, —S(O₂)OR_{A};
R_{A} is H;
R_{B} and R_{C} are independently H or —S(O₂)R_{D};
R_{D} is (C₁-C₆)alkyl, —CF₃, or phenyl;
R₂, R₃, and R₄ are independently H, (C₁-C₆)alkyl, cyano, or halogen;
R₅ is H;
R₆, R₇, R₉, and R₁₀ are independently H, (C₁-C₆)alkoxy, (C₁-C₆)alkyl, cyano, halogen, hydroxy, or hydroxy(C₁-C₆)alkyl;
R₈ is H, (C₁-C₆)alkoxy, (C₁-C₆)alkoxy(C₁-C₆)alkyl, (C₁-C₆)alkyl, (C₁-C₆)alkylcarbonyl, aryl, carboxy(C₁-C₆)alkoxy, (C₃-C₈)cycloalkyl, (C₃-C₈)cycloalkyl(C₁-C₆)alkyl, (C₃-C₈)cycloalkyloxy, halo(C₁-C₆)alkyl, heteroaryl(C₁-C₆)alkoxy, (C₃-C₇)heterocycle, (C₃-C₂)heterocycle(C₁-C₆)alkoxy, (C₃-C₇)heterocyclecarbonyl(C₁-C₆)alkyl, (C₃-C₇)heterocycleoxy, hydroxy(C₁-C₆)alkoxy, hydroxy(C₁-C₆)alkyl, —NR_{M}R_{N}, (NR_{M}R_{N})carbonyl(C₁-C₆)alkyl, or (NR_{M}R_{N})carbonyl(C₁-C₆)alkoxy; wherein the aryl is optionally substituted with 1 substituent that is (C₁-C₆)alkoxy or hydroxy; wherein the halo(C₁-C₆)alkyl is optionally with 1 hydroxy group; wherein the (C₃-C₈)cycloalkyl, (C₃-C₈)cycloalkyl(C₁-C₆)alkyl, and (C₃-C₈)cycloalkyloxy are optionally substituted with 1 substituent that is carboxy, hydroxy, hydroxy(C₁-C₆)alkyl, or (NR_{M}R_{N})carbonyl; and wherein the (C₃-C₇)heterocycle and (C₃-C₇)heterocycle(C₁-C₆)alkoxy are optionally substituted with 1 substituent that is (C₁-C₆)alkoxycarbonyl, (C₁-C₆)alkyl, (C₁-C₆)alkylcarbonyl, (C₁-C₆)alkylsulfonyl, hydroxy, hydroxy(C₁-C₆)alkyl, or oxo; and R_{M} and R_{N} are independently H, (C₁-C₆)alkyl, or (C₁-C₆)alkylcarbonyl; or R_{M} and R_{N} together with the nitrogen they are attached to form a 3 to 8 membered ring.

In some embodiments, the β1-selective AMPK activator is a compound of Formula (II):

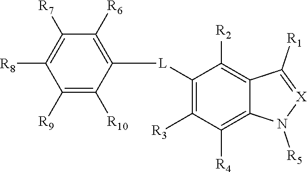

or a pharmaceutically acceptable salt thereof, wherein:
X is N or CH;
L is a bond, O, S, NRA, (C₁-C₆)alkylene, (C₂-C₆)alkenylene, or (C₂-C₆)alkynylene;
R₁ is —C(O)OR_{A}, —C(O)NR_{B}R_{C}, —S(O₂)OR_{A}, —S(O₂)NHC(O)R_{D}, 5-oxo-4,5-dihydro-1,2,4-oxadiazol-3-yl, or 1H-tetrazol-5-yl;
R_{A} is H or (C₁-C₆)alkyl;
R_{B} and R_{C} are independently H, (C₁-C₆)alkyl, or —S(O₂)R_{D};
R_{D} is (C₁-C₈)alkyl, —CF₃, or phenyl, wherein the phenyl is optionally substituted with 1, 2, 3, 4, or 5 substituents that are independently (C₁-C₆)alkoxy, (C₁-C₆)alkyl, cyano, halogen, halo(C₁-C₆)alkoxy, halo(C₁-C₆)alkyl, hydroxy, mercapto, nitro, or NR_{E}R_{F};
R_{E} and R_{F} are independently H or (C₁-C₆)alkyl;
R₂, R₃, and R₄ are independently H, (C₁-C₆)alkoxy, (C₁-C₆)alkyl, (C₁-C₆)alkylthio, carboxy, cyano, halogen, halo(C₁-C₆)alkoxy, halo(C₁-C₆)alkyl, hydroxy, hydroxy(C₁-C₆)alkyl, mercapto, nitro, —NR_{G}R_{H}, or (NR_{G}R_{H})carbonyl; R_{G} and R_{H} are independently H, (C₁-C₆)alkyl, or (C₁-C₆)alkylcarbonyl; R₅ is H or (C₁-C₆)alkyl;
R₆, R₇, R₉, and R₁₀ are independently H, (C₁-C₆)alkoxy, (C₁-C₆)alkoxycarbonyl, (C₁-C₆)alkyl, (C₁-C₆)alkylcarbonyl, (C₁-C₆)alkylthio, carboxy, cyano, halogen, halo(C₁-C₆)alkoxy, halo(C₁-C₆)alkyl, hydroxy, hydroxy(C₁-C₆)alkyl, mercapto, nitro, —NR_{J}R_{K}, or (NR_{J}R_{K})carbonyl; R_{J} and R_{K} are independently H or (C₁-C₆)alkyl;
R₈ is H, (C₁-C₆)alkoxy, (C₁-C₆)alkoxy(C₁-C₆)alkoxy, (C₁-C₆)alkoxy(C₁-C₆)alkyl, (C₁-C₆)alkoxycarbonyl, (C₁-C₆)alkyl, (C₁-C₆)alkylcarbonyl, (C₁-C₆)alkylthio, aryl, aryl(C₁-C₆)alkoxy, aryl(C₁-C₆)alkyl, arylcarbonyl, aryloxy, carboxy, carboxy(C₁-C₆)alkoxy, carboxy(C₁-C₆)alkyl, cyano, (C₃-C₈)cycloalkyl, (C₃-C₈)cycloalkyl(C₁-C₆)alkoxy, (C₃-C₈)cycloalkyl(C₁-C₆)alkyl, (C₃-C₈)cycloalkylcarbonyl, (C₃-C₈)cycloalkyloxy, halogen, halo(C₁-C₆)alkoxy, halo(C₁-C₆)alkyl, heteroaryl, heteroaryl(C₁-C₆)alkoxy, heteroaryl(C₁-C₆)alkyl, heteroarylcarbonyl, heteroaryloxy, (C₃-C₇)heterocycle, (C₃-C₇)heterocycle(C₁-C₆)alkoxy, (C₃-C₇)heterocycle(C₁-C₆)alkyl, (C₃-C₇)heterocyclecarbonyl(C₃-C₇)heterocyclecarbonyl(C₁-C₆)alkyl, (C₃-C₇)heterocycleoxy, hydroxy, hydroxy(C₁-C₆)alkoxy, hydroxy(C₁-C₆)alkyl, mercapto,nitro, —NR_{M}R_{N}, —NR_{M}R_{N}(C₁-C₆))alkoxy, (NR_{M}R_{N})carbonyl, (NR_{M}R_{N})carbonyl(C₁-C₆)alkyl, or (NR_{M}R_{N})carbonyl(C₁-C₆)alkoxy;
wherein the aryl, aryl(C₁-C₆)alkoxy, aryl(C₁-C₆)alkyl, arylcarbonyl, and aryloxy are optionally substituted with 1, 2, 3, 4, or 5 substituents that are independently (C₁-C₆)alkoxy, (C₁-C₆)alkoxycarbonyl, (C₁-C₆)alkyl, (C₁-C₆)alkylcarbonyl, (C₁-C₆)alkylthio, carboxy, cyano, halogen, halo(C₁-C₆)alkoxy, halo(C₁-C₆)alkyl, hydroxy, hydroxy(C₁-C₆)alkyl, mercapto, nitro, —NR_{M}R_{N}, or (NR_{M}R_{N})carbonyl; wherein the halo(C₁-C₆)alkyl is optionally substituted with 1 or 2 hydroxy groups; wherein the (C₃-C₇)cycloalkyl, (C₃-C₇)cycloalkyl(C₁-C₆)alkoxy, (C₃-C₇)cycloalkyl(C₁-C₆)alkyl, (C₃-C₇)cycloalkylcarbonyl, and (C₃-C₇)cycloalkyloxy are optionally substituted with 1, 2, or 3 substituents that are independently (C₁-C₆)alkoxy, (C₁-C₆)alkoxycarbonyl, (C₁-C₆)alkyl, (C₁-C₆)alkylcarbonyl, (C₁-C₆)alkylthio, carboxy, cyano, halogen, halo(C₁-C₆))alkoxy, halo(C₁-C₆)alkyl, hydroxy, hydroxy(C₁-C₅)alkyl, mercapto, nitro, —NR_{M}R_{N}, or (NR_{M}R_{N})carbonyl; wherein the heteroaryl, heteroaryl(C₁-C₆)alkoxy, heteroaryl(C₁-C₆)alkyl, heteroarylcarbonyl, and heteroaryloxy, are optionally substituted with 1, 2, or 3 substituents that are independently (C₁-C₆)alkoxy, (C₁-C₆)alkoxycarbonyl, (C₁-C₆)alkyl, (C₁-C₆)alkylcarbonyl, (C₁-C₆)alkylthio, carboxy, cyano, halogen, halo(C₁-C₆)alkoxy, halo(C₁-C₆))alkyl, hydroxy, hydroxy(C₁-C₆)alkyl, mercapto, nitro, —NR_{M}R_{N}, or (NR_{M}R_{N})carbonyl; and wherein the (C₃-C₇)heterocycle, (C₃-C₇)heterocycle(C₁-C₆)alkoxy, (C₃-C₇)heterocycle(C₁-C₆)alkyl, (C₃-C₇)heterocyclecarbonyl, (C₃-C₇)heterocyclecarbonyl(C₁-C₆)alkyl, and (C₃-C₇)heterocycleoxy, are optionally substituted with 1, 2, or 3 substituents that are independently (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylsulfonyl, (C₁-C₆)alkylthio, carboxy, cyano, halogen, halo(C₁-C₆)alkoxy, halo(C₁-C₆)alkyl, hydroxy, hydroxy(C₁-C₆))alkyl, mercapto, nitro, —NR_{M}R_{N}, (NR_{M}R_{N})carbonyl, or oxo; and R_{M} and R_{N} are independently H, (C₁-C₆)alkyl, or (C₁-C₆)alkylcarbonyl; and R_{M} and R_{N} together with the nitrogen they are attached to form a 3 to 8 membered ring; provided that Formula (II) does not encompass
5-(4-bromophenyl)-1H-indole-3-carboxamide; 5-(2′,6′-dihydroxy-[1,1′-biphenyl]-4-yl)-1H-indole-3-carboxamide; and 5-(2′,6′-dimethoxy-[1,1]biphenyl]-4-yl)-1H-indole-3-carboxamide.

In some embodiments, the β1-selective AMPK activator is a compound of Formula (II):

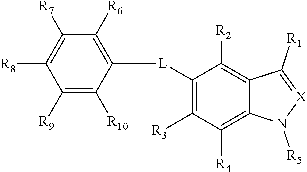

or a pharmaceutically acceptable salt thereof, wherein:
X is CH;
L is a bond;
R₁ is —C(O)OR_{A};
R_{A} is H;
R₂ is H or F;
R₃ is Cl, F, or CN;
R₄ and R₅ are H;
R₆ and R₇ are independently H, F, or methoxy;
R₉ and R₁₀ are H; and
R₈ is (C₃-C₈)cycloalkyl wherein the (C₃-C₈)cycloalkyl
is cyclopropyl or cyclobutyl substituted with hydroxy.

In some embodiments, the β1-selective AMPK activator is a compound of Formula (II) above, selected from the group consisting of:
6-chloro-5-[2-fluoro-4-(1-hydroxycyclobutyl)phenyl]11H-indole-3-carboxylic acid;
6-chloro-5-[3-fluoro-4-(1-hydroxycyclobutyl)phenyl]-1H-indole-3-carboxylic acid; and
6-chloro-5-[4-(1-hydroxycyclobutyl)-3-methoxyphenyl]-1H-indole-3-carboxylic acid;
or a pharmaceutically acceptable salt thereof.

In some embodiments, the β1-selective AMPK activator is Compound (1):

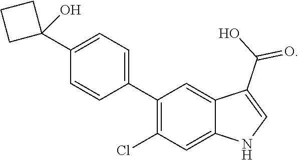 [SMILES: O=C(O)c1c[nH]c2cc(Cl)c(-c3ccc(C4(O)CCC4)cc3)cc12]

(Compound 1), or a pharmaceutically acceptable salt thereof.

In some embodiments, the β1-selective AMPK activator is Compound (2):

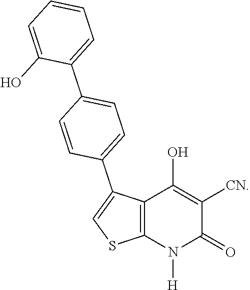 [SMILES: N#Cc1c(O)c2c(-c3ccc(-c4ccccc4O)cc3)csc2[nH]c1=O]

(Compound 2), or a pharmaceutically acceptable salt thereof.

In some embodiments, the compositions and methods described herein may utilize one or more AMPK activators selected from, but not limited to, an AMPK activator described in U.S. Pat. No. 8,080,668, issued Dec. 20, 2011; U.S. Pat. No. 8,119,809, issued Feb. 21, 2012; U.S. Pat. No. 8,273,744, issued Sep. 25, 2012; U.S. Pat. No. 8,329,698, issued Dec. 11, 2012; U.S. Pat. No. 8,329,738, issued Dec. 11, 2012; U.S. Pat. No. 8,563,729, issued Oct. 22, 2013; U.S. Pat. No. 8,569,340, issued Oct. 29, 2013; U.S. Pat. No. 8,604,202, issued Dec. 10, 2013; U.S. Pat. No. 8,809,370, issued Aug. 19, 2014; U.S. Pat. No. 8,980,895, issued Mar. 17, 2015; U.S. Pat. No. 8,980,921, issued Mar. 17, 2015; U.S. Pat. No. 8,987,303, issued Mar. 24, 2015; U.S. Pat. No. 9,174,964, issued Nov. 3, 2015; U.S. Pat. No. 9,284,329, issued Mar. 15, 2016; U.S. Pat. No. 9,365,584, issued Jun. 14, 2016; U.S. Pat. No. 9,394,285, issued Jul. 19, 2016; U.S. Pat. No. 10,377,742, issued Aug. 13, 2019; U.S. Pat. No. 10,941,134, issued Mar. 9, 2021; or U.S. Pat. No. 10,968,186, issued Apr. 6, 2021; The presently disclosed compounds, e.g., any of the compounds disclosed herein that are basic in nature are generally capable of forming a wide variety of different salts with various inorganic and/or organic acids. Although such salts are generally pharmaceutically acceptable for administration to animals and humans, it is often desirable in practice to initially isolate a compound from the reaction mixture as a pharmaceutically unacceptable salt and then simply convert the latter back to the free base compound by treatment with an alkaline reagent, and subsequently convert the free base to a pharmaceutically acceptable acid addition salt. The acid addition salts of the base compounds can be readily prepared using conventional techniques, e.g. by treating the base compound with a substantially equivalent amount of the chosen mineral or organic acid in an aqueous solvent medium or in a suitable organic solvent such as, for example, methanol or ethanol. Upon careful evaporation of the solvent, the desired solid salt is obtained. Presently disclosed compounds that are positively charged, e.g. containing a quaternary ammonium, may also form salts with the anionic component of various inorganic and/or organic acids.

Acids which can be used to prepare pharmaceutically acceptable salts of AMPK activators are those which can form non-toxic acid addition salts, e.g. salts containing pharmacologically acceptable anions, such as chloride, bromide, iodide, nitrate, sulfate or bisulfate, phosphate or acid phosphate, acetate, lactate, citrate or acid citrate, tartrate or bitartrate, succinate, malate, maleate, fumarate, gluconate, saccharate, benzoate, methanesulfonate and pamoate [i.e. 1,1′-methylene-bis-(2-hydroxy-3-naphthoate)]salts.

Presently disclosed compounds that are acidic in nature, e.g. compounds containing a thiol moiety, are generally capable of forming a wide variety of different salts with various inorganic and/or organic bases. Although such salts are generally pharmaceutically acceptable for administration to animals and humans, it is often desirable in practice to initially isolate a compound from the reaction mixture as a pharmaceutically unacceptable salt and then simply convert the latter back to the free acid compound by treatment with an acidic reagent, and subsequently convert the free acid to a pharmaceutically acceptable base addition salt. These base addition salts can be readily prepared using conventional techniques, e.g. by treating the corresponding acidic compounds with an aqueous solution containing the desired pharmacologically acceptable cations, and then evaporating the resulting solution to dryness, e.g. under reduced pressure. Alternatively, they also can be prepared by mixing lower alkanolic solutions of the acidic compounds and the desired alkali metal alkoxide together, and then evaporating the resulting solution to dryness in the same manner as before. In either case, stoichiometric quantities of reagents may be employed in order to ensure completeness of reaction and maximum product yields of the desired solid salt.

Bases which can be used to prepare the pharmaceutically acceptable base addition salts of AMPK activators are those which can form non-toxic base addition salts, e.g. salts containing pharmacologically acceptable cations, such as, alkali metal cations (e.g. potassium and sodium), alkaline earth metal cations (e.g. calcium and magnesium), ammonium or other water-soluble amine addition salts such as N-methylglucamine (meglumine), lower alkanolammonium, and other such bases of organic amines.

The present disclosure further embraces stereoisomers and mixture of stereoisomers of the compounds disclosed herein. Stereoisomers (e.g. cis and trans isomers) and all optical isomers of a presently disclosed compound (e.g. R- and S-enantiomers), as well as racemic, diastereomeric and other mixtures of such isomers are within the scope of the present disclosure.

β1-selective AMPK activators, and pharmaceutical compositions containing them, such as those described herein, are useful in therapy, in particular in the therapeutic treatment of blood disorders, including hemoglobinopathies. Subjects to be treated according to the methods described herein include vertebrates, such as mammals.

A hemoglobinopathy is a condition that involves a mutation in human beta-globin or an expression control sequence thereof, such as sickle cell disease (SCD) or beta-thalassemia.

SCD typically arises from a mutation substituting thymine for adenine in the sixth codon of the beta-chain gene of hemoglobin (i.e., GAG to GTG of the HBB gene). This mutation causes glutamate to valine substitution in position 6 of the Hb beta chain. The resulting Hb, referred to as HbS, has the physical properties of forming polymers under conditions of low oxygen tension. SCD is typically an autosomal recessive disorder.

Subjects with SCD may experience a range of medical complications including acute pain episodes, also known as vaso-occlusive crises or vaso-occlusive episodes, that require hospitalization and may progress to more severe complications such as acute chest syndrome.

SCD is associated with vascular disease and stroke and SCD subjects may experience cerebrovascular accidents including transient ischemic attack, overt strokes and silent cerebral infarctions. Retinopathy and seizures are also associated with SCD. Proliferative sickle cell retinopathy (PSR) is a frequent vision-threatening complication in sickle cell anemia, leading to visual impairment. In PSR, the blood vessels become blocked and divert away from the retina causing the retina to starve and die, leading to vision loss.

Subjects with SCD may experience both chronic and acute complications including bone pain crisis as a complication of vaso-occlusive pain, bone and bone marrow infarction, osteonecrosis and vascular necrosis. Subjects with SCD may experience chronic and acute cardiopulmonary complications including acute chest syndrome (ACS), pulmonary hypertension and left-sided heart disease. SCD subjects may experience chronic and acute reticuloendothelial complications including splenic sequestration, which is more prevalent in subjects who have had a first acute pain episode. Splenic sequestration can result in worsened anemia in SCD subjects.

Subjects with SCD may experience chronic and acute gastrointestinal and urogenital complications including cholelithiasis, acute cholecystitis, biliary sludge, acute choledocholithiasis and gallstones. Urogenital complications, including renal dysfunction, may occur at an early age and lead to chronic renal failure. In male subjects with SCD a priapism may occurs as a severe urogenital complication.

Although children with SCD may or may not experience a vaso-occlusive crisis before they reach adolescence, even infants with SCD may develop symptoms. Infants with SCD may develop a syndrome that develops suddenly and lasts several weeks called hand-foot syndrome. Hand-foot syndrome is a dactylitis that presents as exquisite pain and soft tissue swelling of the dorsum of the hands and feet.

β-Thalassemia are a group of inherited blood disorders caused by a variety of mutational mechanisms that result in a reduction or absence of synthesis of β-globin and leading to accumulation of aggregates of unpaired, insoluble α-chains that cause ineffective erythropoiesis, accelerated red cell destruction, and severe anemia. Subjects with beta-thalassemia exhibit variable phenotypes ranging from severe anemia to clinically asymptomatic individuals. The genetic mutations present in β-thalassemia are diverse, and can be caused by a number of different mutations. The mutations can involve a single base substitution or deletions or inserts within, near or upstream of the β globin gene. For example, mutations occur in the promoter regions preceding the beta-globin genes or cause production of abnormal splice variants. β⁰ is used to indicate a mutation or deletion which results in no functional β globin being produced. β⁺ is used to indicate a mutation in which the quantity or β globin is reduced or in which the β-globin produced has a reduced functionality. Examples of β-thalassemia include thalassemia minor, thalassemia intermedia, and thalassemia major. β-Thalassemia minor refers to thalassemia where only one of β-globin alleles bears a mutation. Individuals typically suffer from microcytic anemia. Detection usually involves lower than normal MCV value (<80 fL) plus an increase in fraction of hemoglobin A2 (>3.5%) and a decrease in fraction of hemoglobin A (<97.5%). Genotypes can be β⁺/β or β⁰/s. β-Thalassemia intermedia refers to a β-thalassemia intermediate between the major and minor forms.

Affected individuals can often manage a normal life but may need occasional transfusions, e.g., at times of illness or pregnancy, depending on the severity of their anemia. Genotypes can be β⁺/β⁺ or β⁰/β.

β-Thalassemia major refers to a β-thalassemia where both β-globin alleles have thalassemia mutations. This is a severe microcytic, hypochromic anemia. If left untreated, it causes anemia, splenomegaly, and severe bone deformities and typically leads to death before age 20. Treatment consists of periodic blood transfusion; splenectomy if splenomegaly is present, and treatment of transfusion-caused iron overload. Cure is possible by bone marrow transplantation. Genotypes include β⁺/β⁰ or/or β⁺/β⁺. Mediterranean anemia or Cooley's anemia has a genotype of β⁰/β⁰ so that no hemoglobin A is produced. It is the most severe form of β-thalassemia.

Although carriers of sickle cell trait do not suffer from SCD, individuals with one copy of HbS and one copy of a gene that codes for another abnormal variant of hemoglobin, such as HbC or Hb beta-thalassemia, typically will have a less severe form of sickle cell disease. For example, another specific defect in β-globin causes another structural variant, hemoglobin C (HbC). Hemoglobin C (abbreviated as Hb C or HbC) is an abnormal hemoglobin in which substitution of a glutamic acid residue with a lysine residue at the 6th position of the β-globin chain has occurred. A subject that is a double heterozygote for HbS and HbC (HbSC disease) is typically characterized by symptoms of moderate clinical severity.

Another common structural variant of β-globin is hemoglobin E (HbE). HbE is an abnormal hemoglobin in which substitution of a glutamic acid residue with a lysine residue at the 26th position of the β-globin chain has occurred. A subject that is a double heterozygote for HbS and HbE has HbS/HbE syndrome, which usually causes a phenotype similar to HbS/b+ thalassemia, discussed below.

A subject that is a double heterozygote for HbS and 30 thalassemia (i.e., HbS/β⁰ thalassemia) can suffer symptoms clinically indistinguishable from sickle cell anemia.

A subject that is a double heterozygote for HbS and μ⁺ thalassemia (i.e., HbS/β⁰ thalassemia) can have mild-to-moderate severity of clinical symptoms with variability among different ethnicities.

Rare combinations of HbS with other abnormal hemoglobins include HbD Los Angeles, G-Philadelphia, HbO Arab, and others.

In some embodiments, the β1-selective AMPK activators are used to treat a hemoglobinopathy, such as SCD or thalassemia (e.g. β-thalassemia), including those that involve a mutation in human β-globin or an expression control sequence thereof, as described above. Accordingly, provided herein are methods of treating a hemoglobinopathy comprising administering an effective amount of a β1-selective AMPK activator to a patient in need thereof. In some aspects, the β1-selective AMPK activator is a compound of Formula (I) or a pharmaceutically acceptable salt thereof. In some aspects, the β1-selective AMPK activator is a compound of Formula (II) or a pharmaceutically acceptable salt thereof. In some aspects, the β1-selective AMPK activator is Compound 1 or Compound 2, or a pharmaceutically acceptable salt thereof.

In some embodiments, the β1-selective AMPK activators are used to treat a subject with an HbS/β⁰ genotype, an HbS/μ⁺ genotype, an HBSC genotype, an HbS/HbE genotype, an HbD Los Angeles genotype, a G-Philadelphia genotype, or an abHbO Arab genotype.

In some embodiments, the β1-selective AMPK activators are administered to a subject in need thereof in an effective amount to treat one or more symptoms of sickle cell disease, a thalassemia (e.g. β-thalassemia), or a related disorder. In subjects with sickle cell disease, or a related disorder, physiological changes in RBCs can result in a disease with the following signs: (1) hemolytic anemia; (2) vaso-occlusive crisis; and (3) multiple organ damage from microinfarcts, including heart, skeleton, spleen, and central nervous system. Thalassemia can include symptoms such as anemia, fatigue and weakness, pale skin or jaundice (yellowing of the skin), protruding abdomen with enlarged spleen and liver, dark urine, abnormal facial bones and poor growth, and poor appetite.

Retinopathy due to SCD can also be treated by administering an effective amount of 01-AMPK activator. Sickle retinopathy occurs when the retinal blood vessels get occluded by sickle red blood cells and the retina becomes ischemic, angiogenic factors are made in retina. In sickle cell disease, this occurs mostly in the peripheral retina, which does not obscure vision at first. Eventually, the entire peripheral retina of the sickle cell patient becomes occluded and many neovascular formations occur. Administration of a β1-selective AMPK activator can reduce or inhibit the formation of occlusions in the peripheral retina of a sickle cell patient.

In some embodiments, the β1-selective AMPK activators are used to increase HbF expression in a patient in need thereof.

Accordingly, provided herein are methods of increasing HbF expression comprising administering an effective amount of a β1-selective AMPK activator to a patient in need thereof. In some aspects, the β1-selective AMPK activator is a compound of Formula (I) or a pharmaceutically acceptable salt thereof. In some aspects, the β1-selective AMPK activator is a compound of Formula (II) or a pharmaceutically acceptable salt thereof. In some aspects, the β1-selective AMPK activator is Compound 1 or Compound 2, or a pharmaceutically acceptable salt thereof.

In some embodiments, the β1-AMPK activators are used to decrease inflammation in a patient with a β-hemoglobinopathy.

Accordingly, provided herein are decreasing inflammation in β-hemoglobinopathy comprising administering an effective amount of a β1-selective AMPK activator to a patient in need thereof. In some aspects, the β1-selective AMPK activator is a compound of Formula (I) or a pharmaceutically acceptable salt thereof. In some aspects, the β1-selective AMPK activator is a compound of Formula (II) or a pharmaceutically acceptable salt thereof. In some aspects, the β1-selective AMPK activator is Compound 1 or Compound 2, or a pharmaceutically acceptable salt thereof.

In some embodiments, the β1-AMPK activators are used to decrease oxidative stress in a patient with a β-hemoglobinopathy.

Accordingly, provided herein are decreasing oxidative stress in β-hemoglobinopathy comprising administering an effective amount of a β1-selective AMPK activator to a patient in need thereof. In some aspects, the β1-selective AMPK activator is a compound of Formula (I) or a pharmaceutically acceptable salt thereof. In some aspects, the β1-selective AMPK activator is a compound of Formula (II) or a pharmaceutically acceptable salt thereof. In some aspects, the β1-selective AMPK activator is Compound 1 or Compound 2, or a pharmaceutically acceptable salt thereof.

In some embodiments, the β1-selective AMPK activator is administered in combination with hydroxyurea.

Accordingly, provided herein is a method of treating a hemoglobinopathy comprising administering an effective amount of a β1-selective AMPK activator to a patient in need thereof in combination with an effective amount of hydroxyurea. In some aspects, the β1-selective AMPK activator is a compound of Formula (I) or a pharmaceutically acceptable salt thereof. In some aspects, the β1-selective AMPK activator is a compound of Formula (II) or a pharmaceutically acceptable salt thereof. In some aspects, the β1-selective AMPK activator is Compound 1 or a pharmaceutically acceptable salt thereof. In some aspects, the β1-selective AMPK activator is Compound 2, or a pharmaceutically acceptable salt thereof.

In another aspect, provided herein are methods of treating a hemoglobinopathy comprising administering an effective amount of a combination of a β1-selective AMPK activator and hydroxyurea to a patient in need thereof. In some aspects, the β1-selective AMPK activator is a compound of Formula (I) or a pharmaceutically acceptable salt thereof. In some aspects, the β1-selective AMPK activator is a compound of Formula (II) or a pharmaceutically acceptable salt thereof. In some aspects, the β1-selective AMPK activator is Compound 1 or a pharmaceutically acceptable salt thereof. In some aspects, the β1-selective AMPK activator is Compound 2 or a pharmaceutically acceptable salt thereof.

### Pharmaceutical Compositions

The present disclosure also provides pharmaceutical compositions comprising at least one β1-selective AMPK activator as described herein and at least one pharmaceutically acceptable excipient, e.g. for use according to the methods disclosed herein. The pharmaceutically acceptable excipient can be any such excipient known in the art including those described in, for example, Remington's Pharmaceutical Sciences, Mack Publishing Co. (A. R. Gennaro edit. 1985). Pharmaceutical compositions of the β1-selective AMPK activator may be prepared by conventional means known in the art including, for example, mixing at least one β1-selective AMPK activator with a pharmaceutically acceptable excipient.

Thus, in one aspect the present disclosure provides a pharmaceutical dosage form comprising a β1-selective AMPK activator as described herein and a pharmaceutically acceptable excipient, wherein the dosage form is formulated to provide, when administered (e.g. when administered orally), an amount of said compound sufficient to treat a disease or disorder as described herein.

A pharmaceutical composition or dosage form of the invention can include an agent and another carrier, e.g. compound or composition, inert or active, such as a detectable agent, label, adjuvant, diluent, binder, stabilizer, buffers, salts, lipophilic solvents, preservative, adjuvant or the like. Carriers also include pharmaceutical excipients and additives, for example, proteins, peptides, amino acids, lipids, and carbohydrates (e.g. sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1 to 99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this invention, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

Carriers which may be used include a buffer or a pH adjusting agent; typically, the buffer is a salt prepared from an organic acid or base. Representative buffers include organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. Additional carriers include polymeric excipients/additives such as polyvinylpyrrolidones, ficolls (a polymeric sugar), dextrates (e.g. cyclodextrins, such as 2-hydroxypropyl-β-cyclodextrin), polyethylene glycols, flavoring agents, antimicrobial agents, sweeteners, antioxidants, antistatic agents, surfactants (e.g. polysorbates such as “TWEEN 20” and “TWEEN 80”), lipids (e.g. phospholipids, fatty acids), steroids (e.g. cholesterol), and chelating agents (e.g. EDTA).

The β1-selective AMPK activators and pharmaceutical compositions can be used in an animal or human. Thus, a β1-selective AMPK activator can be formulated as an active ingredient in a pharmaceutical composition for oral, buccal, parenteral (e.g. intravenous, intramuscular or subcutaneous), topical, rectal or intranasal administration or in a form suitable for administration by inhalation or insufflation. In particular embodiments, the β1-AMPK activator or pharmaceutical composition is formulated for systemic administration, e.g. via a non-parenteral route. In one embodiment, the β1-AMPK activator or pharmaceutical composition is formulated for oral administration, e.g. in solid form. Such modes of administration and the methods for preparing appropriate pharmaceutical compositions are described, for example, in Gibaldi's Drug Delivery Systems in Pharmaceutical Care (1st ed., American Society of Health-System Pharmacists 2007).

The pharmaceutical compositions can be formulated so as to provide slow, extended, or controlled release of the active ingredient therein using, for example, hydroxypropyl methyl cellulose in varying proportions to provide the desired release profile, other polymer matrices, liposomes and/or microspheres. The pharmaceutical compositions can also optionally contain opacifying agents and may be of a composition that releases the active ingredient(s) only, or preferentially, in a certain portion of the gastrointestinal tract, optionally, in a delayed manner, e.g. by using an enteric coating. Examples of embedding compositions include polymeric substances and waxes. The active ingredient can also be in micro-encapsulated form, if appropriate, with one or more pharmaceutically acceptable carriers, excipients, or diluents well known in the art (see, e.g., Remington's). The β1-selective AMPK activator may be formulated for sustained delivery according to methods well known to those of ordinary skill in the art. Examples of such formulations can be found in U.S. Pat. Nos. 3,119,742; 3,492,397; 3,538,214; 4,060,598; and 4,173,626.

In solid dosage forms for oral administration (e.g. capsules, tablets, pills, dragees, powders, granules and the like), the active ingredient is mixed with one or more pharmaceutically acceptable carriers, excipients, or diluents, such as sodium citrate or dicalcium phosphate, and/or any of the following: (1) fillers or extenders, such as starches, lactose, sucrose, glucose, mannitol, microcrystalline cellulose, calcium phosphate and/or silicic acid; (2) binders, such as, for example, carboxymethylcellulose, alginates, gelatin, pregelatinized maize starch, polyvinyl pyrrolidone, hydroxypropyl methylcellulose, sucrose and/or acacia; (3) humectants, such as glycerol; (4) disintegrating agents, such as agar-agar, calcium carbonate, sodium starch glycolate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, sodium lauryl sulphate, acetyl alcohol and glycerol monostearate; (8) absorbents, such as kaolin and bentonite clay; (9) lubricants, such as talc, silica, calcium stearate, magnesium stearate, solid polyethylene glycols, sodium lauryl sulfate, and mixtures thereof, and (10) coloring agents. In the case of capsules, tablets, and pills, the pharmaceutical compositions can also comprise buffering agents. Solid compositions of a similar type can also be prepared using fillers in soft and hard-filled gelatin capsules, and excipients such as lactose or milk sugars, as well as high molecular weight polyethylene glycols and the like.

A tablet can be made by compression or molding, optionally with one or more accessory ingredients. Compressed tablets can be prepared using binders (for example, gelatin or hydroxypropyl methyl cellulose), lubricants, inert diluents, preservatives, disintegrants (for example, sodium starch glycolate or cross-linked sodium carboxymethyl cellulose), surface-actives, and/or dispersing agents. Molded tablets can be made by molding in a suitable machine a mixture of the powdered active ingredient moistened with an inert liquid diluent. The tablets and other solid dosage forms, such as dragees, capsules, pills, and granules, can optionally be scored or prepared with coatings and shells, such as enteric coatings and other coatings well known in the art.

In some embodiments, the pharmaceutical compositions are administered orally in a liquid form. Liquid dosage forms for oral administration of an active ingredient include pharmaceutically acceptable emulsions, microemulsions, solutions, suspensions, syrups and elixirs. Liquid preparations for oral administration may be presented as a dry product for constitution with water or other suitable vehicle before use. In addition to the active ingredient, the liquid dosage forms can contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (e.g. cottonseed, groundnut, corn, germ, olive, castor and sesame oils), glycerol, tetrahydrofuryl alcohol, polyethylene glycols and fatty acid esters of sorbitan, and mixtures thereof. In addition to inert diluents, the liquid pharmaceutical compositions can include adjuvants such as wetting agents, emulsifying and suspending agents, sweetening, flavoring, coloring, perfuming and preservative agents, and the like. Suspensions, in addition to the active ingredient(s) can contain suspending agents such as, but not limited to, ethoxylated isostearyl alcohols, polyoxyethylene sorbitol and sorbitan esters, microcrystalline cellulose, aluminum metahydroxide, bentonite, agar-agar and tragacanth, and mixtures thereof. Suitable liquid preparations may be prepared by conventional means with a pharmaceutically acceptable additive(s) such as a suspending agent (e.g. sorbitol syrup, methyl cellulose or hydrogenated edible fats); emulsifying agent (e.g. lecithin or acacia); non-aqueous vehicle (e.g. almond oil, oily esters or ethyl alcohol); and/or preservative (e.g. methyl or propyl p-hydroxybenzoates or sorbic acid). The active ingredient(s) can also be administered as a bolus, electuary, or paste.

For buccal administration, the composition may take the form of tablets or lozenges formulated in a conventional manner.

In some embodiments, the pharmaceutical compositions are administered by non-oral means such as by topical application, transdermal application, injection, and the like. In related embodiments, the pharmaceutical compositions are administered parenterally by injection, infusion, or implantation (e.g. intravenous, intramuscular, intra-arterial, subcutaneous, and the like).

The β1-selective AMPK activator may be formulated for parenteral administration by injection, including using conventional catheterization techniques or infusion. Formulations for injection may be presented in unit dosage form, e.g. in ampules or in multi-dose containers, with an added preservative. The compositions may take such forms as suspensions, solutions or emulsions in oily or aqueous vehicles, and may contain a formulating agent such as a suspending, stabilizing and/or dispersing agent recognized by those of skill in the art. Alternatively, the active ingredient may be in powder form for reconstitution with a suitable vehicle, e.g. sterile pyrogen-free water, before use.

The pharmaceutical compositions may be administered directly to the central nervous system. Accordingly, in certain embodiments the compositions are administered directly to the central nervous system so as to avoid the blood brain barrier. In some embodiments, the composition can be administered via direct spinal cord injection. In some embodiments, the composition is administered by intrathecal injection. In some embodiments, the composition is administered via intracerebroventricular injection. In some embodiments, the composition is administered into a cerebral lateral ventricle. In some embodiments, the composition is administered into both cerebral lateral ventricles. In additional embodiments, the composition is administered via intrahippocampal injection. The compositions may be administered in one injection or in multiple injections. In other embodiments, the composition is administered to more than one location (e.g. to two sites in the central nervous system).

The pharmaceutical compositions can be in the form of sterile injections. The pharmaceutical compositions can be sterilized by, for example, filtration through a bacteria-retaining filter, or by incorporating sterilizing agents in the form of sterile solid compositions which can be dissolved in sterile water, or some other sterile injectable medium immediately before use. To prepare such a composition, the active ingredient is dissolved or suspended in a parenterally acceptable liquid vehicle. Exemplary vehicles and solvents include, but are not limited to, water, water adjusted to a suitable pH by addition of an appropriate amount of hydrochloric acid, sodium hydroxide or a suitable buffer, 1,3-butanediol, Ringer's solution and isotonic sodium chloride solution. The pharmaceutical composition can also contain one or more preservatives, for example, methyl, ethyl or n-propyl p-hydroxybenzoate. To improve solubility, a dissolution enhancing or solubilizing agent can be added or the solvent can contain 10-60% w/w of propylene glycol or the like.

The pharmaceutical compositions can contain one or more pharmaceutically acceptable sterile isotonic aqueous or nonaqueous solutions, dispersions, suspensions or emulsions, or sterile powders, which can be reconstituted into sterile injectable solutions or dispersions just prior to use. Such pharmaceutical compositions can contain antioxidants; buffers; bacteriostats; solutes, which render the formulation isotonic with the blood of the intended recipient; suspending agents; thickening agents; preservatives; and the like.

Examples of suitable aqueous and nonaqueous carriers, which can be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. In some embodiments, in order to prolong the effect of an active ingredient, it is desirable to slow the absorption of the compound from subcutaneous or intramuscular injection. This can be accomplished by the use of a liquid suspension of crystalline or amorphous material having poor water solubility. The rate of absorption of the active ingredient then depends upon its rate of dissolution which, in turn, can depend upon crystal size and crystalline form. Alternatively, delayed absorption of a parenterally-administered active ingredient is accomplished by dissolving or suspending the compound in an oil vehicle. In addition, prolonged absorption of the injectable pharmaceutical form can be brought about by the inclusion of agents that delay absorption such as aluminum monostearate and gelatin.

Controlled release parenteral compositions can be in form of aqueous suspensions, microspheres, microcapsules, magnetic microspheres, oil solutions, oil suspensions, emulsions, or the active ingredient can be incorporated in biocompatible carrier(s), liposomes, nanoparticles, implants or infusion devices. Materials for use in the preparation of microspheres and/or microcapsules include, but are not limited to, biodegradable/bioerodible polymers such as polyglactin, poly-(isobutyl cyanoacrylate), poly(2-hydroxyethyl-L-glutamine) and poly(lactic acid). Biocompatible carriers which can be used when formulating a controlled release parenteral formulation include carbohydrates such as dextrans, proteins such as albumin, lipoproteins or antibodies. Materials for use in implants can be non-biodegradable, e.g. polydimethylsiloxane, or biodegradable such as, e.g., poly(caprolactone), poly(lactic acid), poly(glycolic acid) or poly(ortho esters).

For topical administration, a β1-selective AMPK activator may be formulated as an ointment, cream, or liquid eye drops. A β1-selective AMPK activator may also be formulated in rectal compositions such as suppositories or retention enemas, e.g. containing conventional suppository bases such as cocoa butter or other glycerides.

For intranasal administration or administration by inhalation, a β1-selective AMPK activator may be conveniently delivered in the form of a solution or suspension from a pump spray container that is squeezed or pumped by the patient or as an aerosol spray presentation from a pressurized container or a nebulizer, with the use of a suitable propellant, e.g. dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the β1-selective AMPK activator. Capsules and cartridges (made, for example, from gelatin) for use in an inhaler or insufflator may be formulated containing a powder mix of a β1-selective AMPK activator and a suitable powder base such as lactose or starch.

Having been generally described herein, the follow non-limiting examples are provided to further illustrate this invention.

### EXAMPLES

### Example 1—Estimation of Major Isoforms in Single Bone Marrow Hematopoietic Cells Shows the Predominant Expression of β1 Isoform and Quasi-Absence of β2 Isoform in Erythroid Lineage

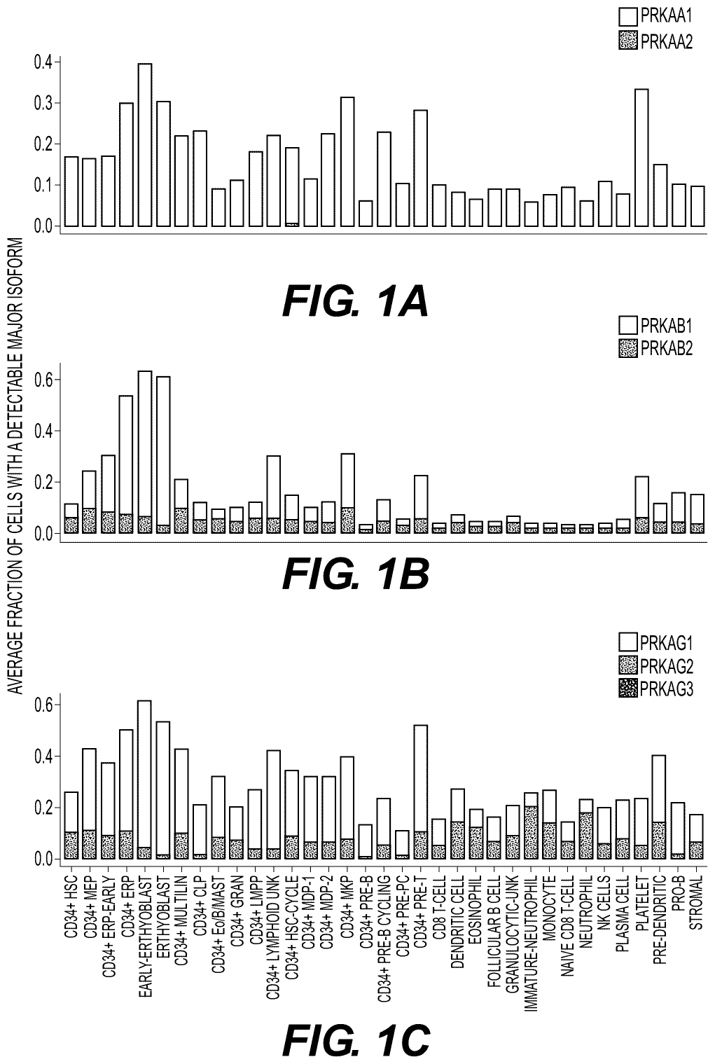

Publicly available single-cell bone marrow gene expression data from 8 independent donors was downloaded from the Human Cell Atlas (https://preview.data.humancellatlas.org/). Cell types were assigned to each bar codes (single cells) as previously (http://www.altanalyze.org/ICGS/HCA/Viewer.php; Hay et al. 2018). The major AMPK isoform in each single cell was then estimated separately for each alpha (a), beta (p), and gamma (7) subunits based on the gene with highest UMI count. No gene was assigned in case of ties between genes. The fraction of cells with each major isoform was averaged across the 8 donors. The R package ggpubr was used for data visualization. The major α-AMPK isoform from early stage CD34+ cells until late erythroblasts is α1-AMPK (FIG. 1A). Both β1-AMPK and β2-AMPK are expressed in early stage CD34+ cells. Expression of β2-AMPK is slightly greater than β1-AMPK in most primitive CD34+ HSCs. During differentiation, expression of β1-AMPK increases and expression β2-AMPK decreases, as reflected by the increasing fraction of cells with β1-AMPK as the most expressed subunit. By the early erythroblast and erythroblast stage β1-AMPK is the major isoform and the ratio of β1-AMPK to β2-AMPK increases greatly from the ratio in early CC34+ cells (FIG. 1B). The 71-AMPK isoform is the major isoform and increases from early stage CD34+ cells until late erythroblasts with lower expression of γ2-AMPK that decreases further during differentiation and very low expression of γ3-AMPK (FIG. 1C).

### Example 2—Induction of Fetal Hemoglobin HbF by “Compound 1” in Human CD34+ Cells During Differentiation In Vitro

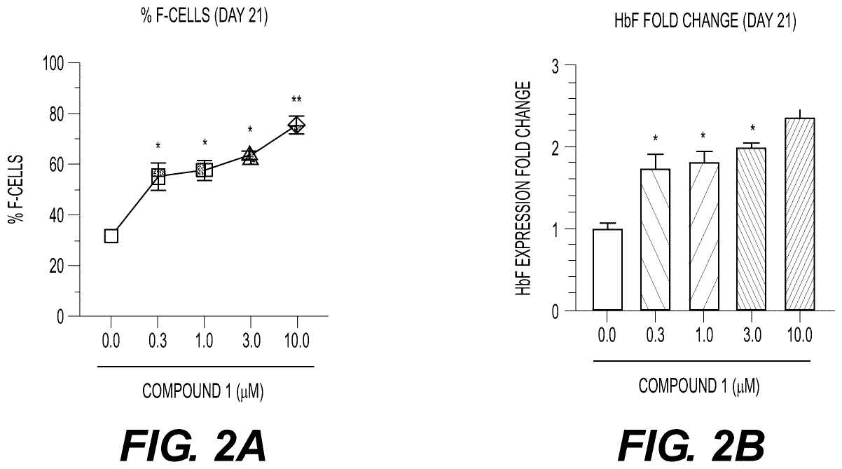

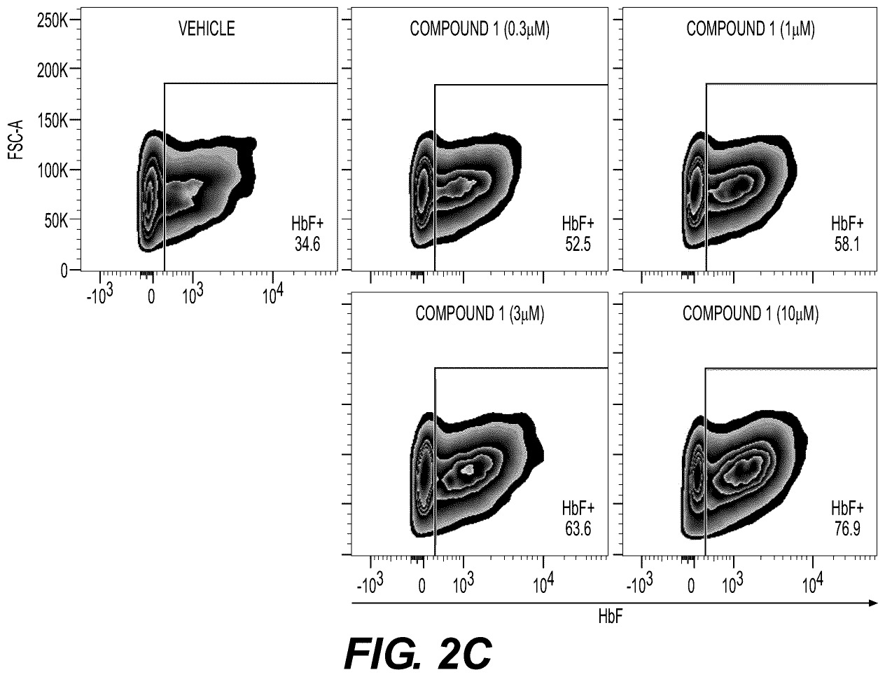

Mobilized CD34+ Human Stem/Progenitor Cells (HSPC) from healthy individuals were cultured for 3 days in a maintenance media consisting of X-VIVO 10 (VWR), 100 U/mL penicillin-streptomycin (ThermoFisher), 2 mM L-glutamine (Fisher Scientific), 100 ng/mL Recombinant Human Stem Cell Factor (SCF), 100 ng/mL Recombinant Human Thrombopoietin (TPO) and 100 ng/mL Recombinant Human Flt-3 Ligand (Flt-3L) (all three from ThermoFisher). Cells were differentiated into erythroid cells using a three-step differentiation protocol developed by the Luc Douay group (Giarratana et al. 2005). In brief, CD34+ cells were cultured for 7 days in Step 1 media, consisting of Iscove's modified Dulbecco's medium (IMDM) (ThermoFisher) supplemented with 1× GlutaMAX, 100 U/mL penicillin-streptomycin (ThermoFisher), 5% human AB+ plasma, 330 ug/mL human holo-transferrin, 10 ug/mL human insulin, 2 U/mL heparin, 1 uM/mL hydrocortisone (Sigma-Aldrich), 3 U/mL recombinant human erythropoietin (EPO) (ThermoFisher), 100 ng/mL SCF (ThermoFisher) and 5 ng/mL interleukin 3 (IL-3) (Sigma-Aldrich). On day 7, cells were transferred to step 2 media, a step 1 media without hydrocortisone and IL-3, and cultured for 3-4 days. Then cells were cultured for 8-9 days in step 3 media, a step 2 media without SCF. During the entire process of differentiation, mobilized CD34+ HSPC were exposed to β1-selective AMPK activator “Compound 1”. To determine the percentage of HbF-positive cells (F-cells), differentiate cells were fixed and permeabilized using a fixation kit (ThermoFisher). Cells were stained with PE-Cy7-conjugated anti-CD235 or PE-conjugated anti-CD71 antibodies. HbF levels were detected using Allophycocyanin (APC)-conjugated anti-HbF antibody (ThermoFisher). The acquisition of stained cells was performed on BD FACSCanto™ and the analysis was run using FlowJo™ Software. Data shows the frequency of F-cells increased when CD34+ cells were exposed to “Compound 1” in a dose-response manner, compared to vehicle (DMSO), after 21 days of differentiation (FIG. 2A). The increase of HbF+ cells is about 2-fold when cells are treated with “Compound 1” compared to DMSO (FIG. 2B).

### Example 3—AMPK Phosphorylation by “Compound 1” in Human CD34+ does not Affect CD34+ Cells Maturation to Erythrocytes In Vitro

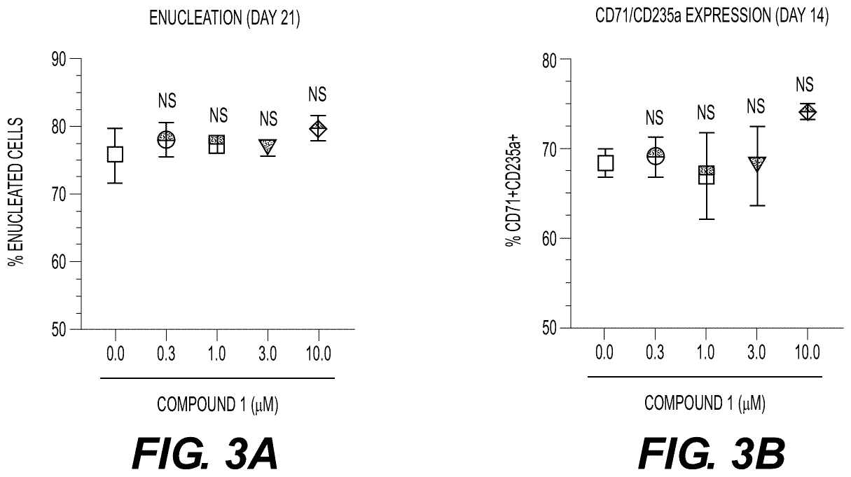

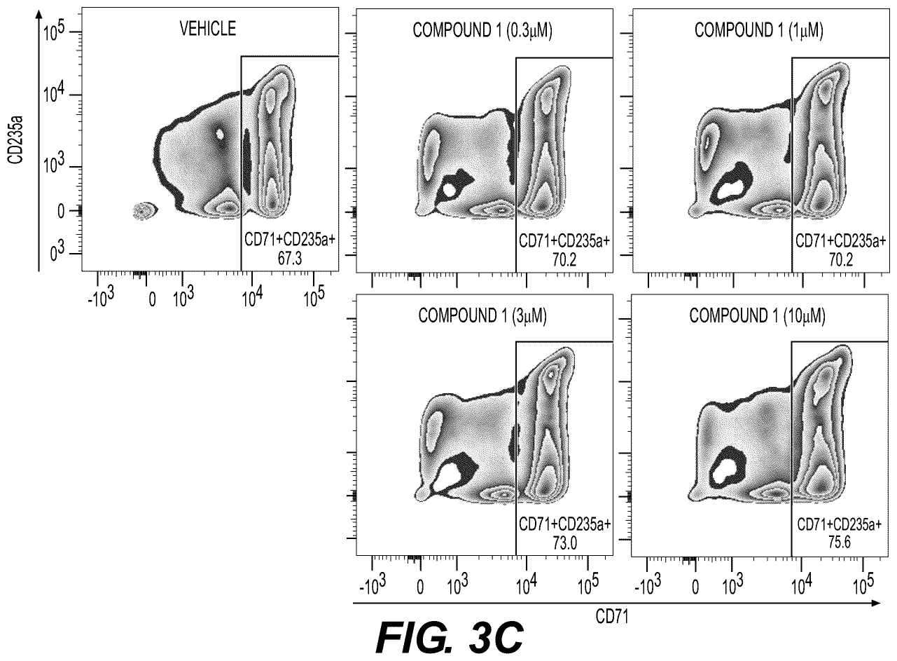

During human CD34+ cells differentiation, maturation is measured by quantification of enucleation and expression of markers CD71 and CD235a, and assessed by flowcytometry. To determine the enucleation rate of the erythroid differentiated cells at day 21, cells were stained using NucRed (living cells marker). As well, cells were stained for markers CD235a and CD71. The acquisition was performed on BD FACSCanto™ and the analysis was done using FlowJo™ Software. Data shows there was no effect of “Compound 1” on the enucleation of CD34+ cells after 21 days of differentiation (FIG. 3A). As well, the expression of differentiation markers CD71 and CD235a was unchanged in presence of “Compound 1” after 14 days of differentiation (FIG. 3B).

### Example 4—Induction of Fetal Hemoglobin HbF by β1-Selective AMPK Activators “Compound 1” and “Compound 2”, and by Pan-AMPK Activators “Compound 3” and “Compound 4” in Human CD34+ Cells During Differentiation In Vitro

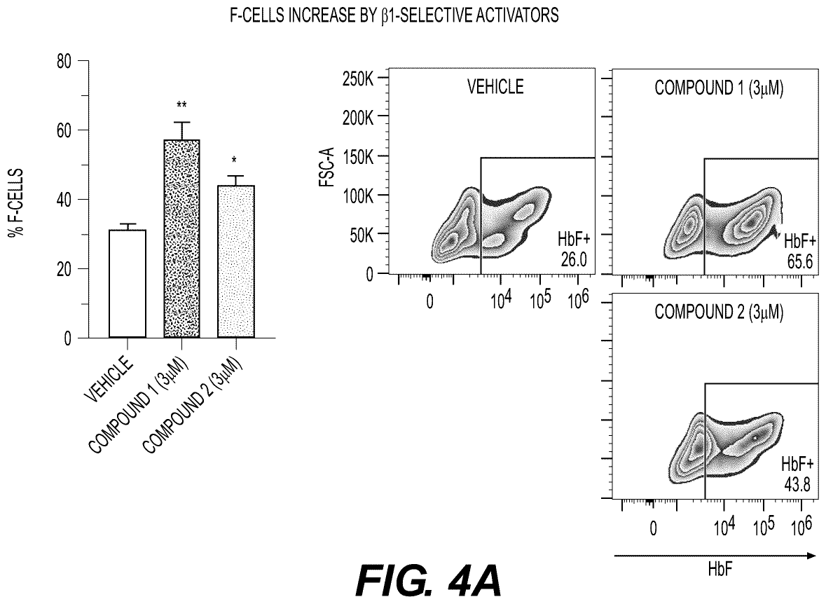

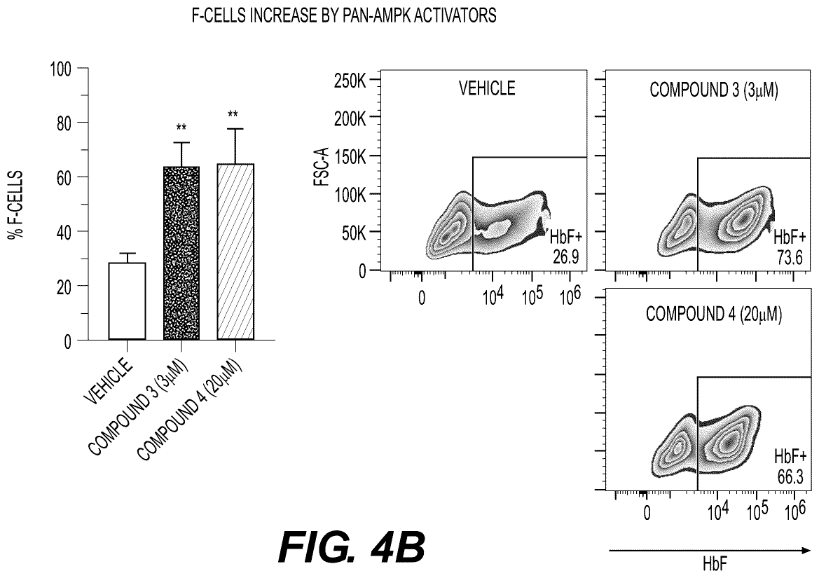

Human CD34+ cells were exposed to β1-selective AMPK activators and pan-AMPK activators during the 21-day differentiation process. To assess fetal hemoglobin expression level, cells were fixed and stained with CD235a, CD71 and fetal hemoglobin antibodies for flowcytometry analysis. Data show an increase of F-cells frequency in differentiated erythroid cells when cells were exposed to β1-selective AMPK activators “Compound 1” and “Compound 2” (FIG. 4A) and to pan-AMPK activators “Compound 3” and “Compound 4” (FIG. 4B).

### Example 5-“Compound 1” and Hydroxyurea Combination Shows Additive Effect on Fetal Hemoglobin Induction in Human CD34+ Cells During Differentiation In Vitro

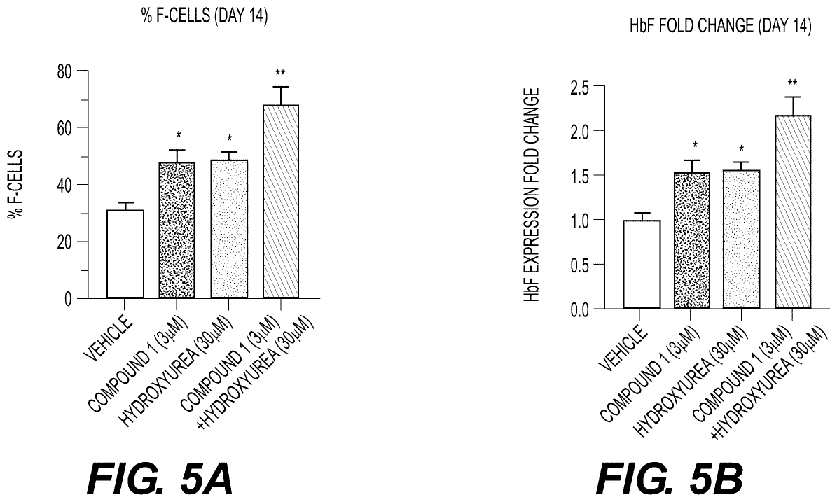

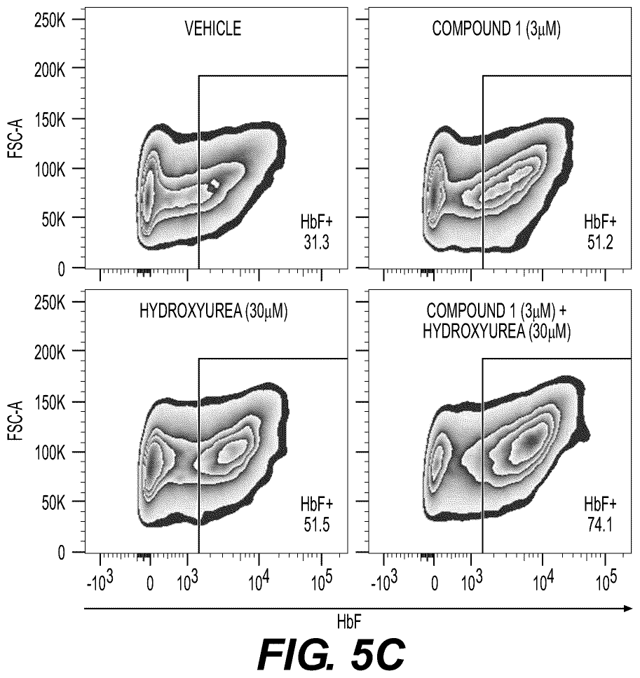

To assess the effect of the combination of Hydroxyurea (Sigma-Aldrich) and “Compound 1”, human CD34+ cells were incubated with “Compound 1” or Hydroxyurea alone, or a combination of both during the differentiation process. After 14 days of differentiation, cells were fixed and stained with CD235a, CD71 and fetal hemoglobin antibodies for flowcytometry analysis. Data show that the combination of Hydroxyurea and “Compound 1” leads to a synergetic effect in fetal hemoglobin induction compared to “Compound 1” or Hydroxyurea alone (FIG. 5A). The fold change in fetal hemoglobin expression is 1,5-fold for “Compound 1” and Hydroxyurea alone, and 2-fold for a combination of both compared to control (Vehicle as DMSO) (FIG. 5B).

### Example 6—Induction of Fetal Hemoglobin HbF by β1-Selective and Pan-AMPK Activators in Human CD34+ Cells from Sickle Cell Donors During Differentiation In Vitro

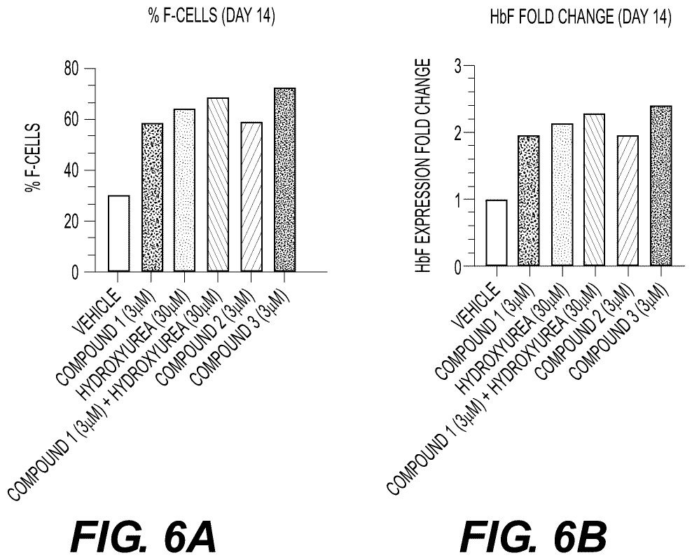

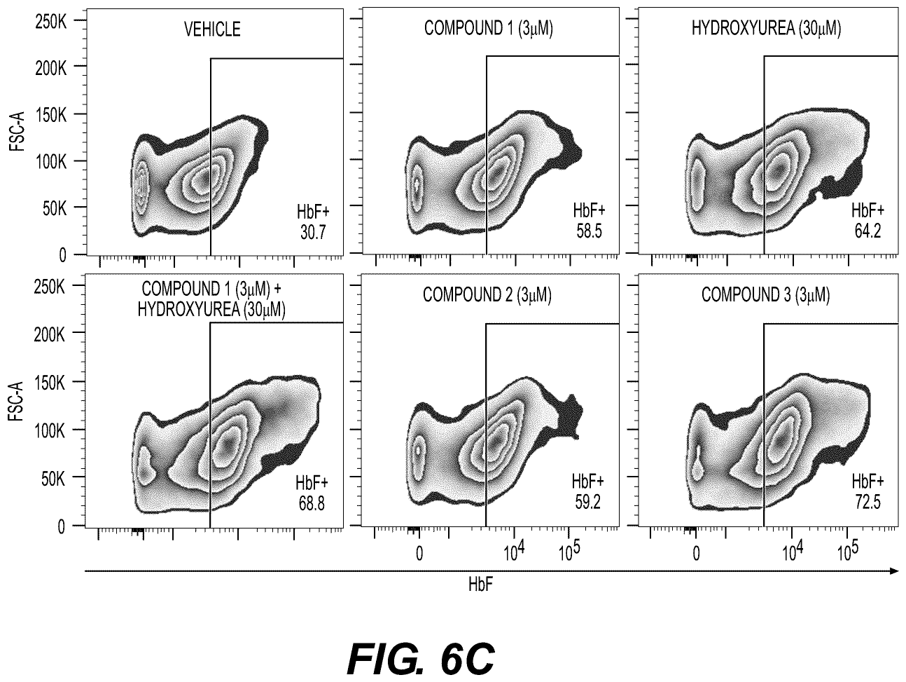

To measure the effect of AMPK activators on fetal hemoglobin induction in CD34+ cells from patients with sickle cell disease, circulating CD34+ progenitor cells were isolated from total blood obtained from a sickle cell patients, by performing a positive selection for CD34+ cells with magnetic beads (Miltenyi Biotec). Purified CD34+ cells were differentiated for 14 days. After differentiation, cells were fixed and stained with CD235a, CD71 and fetal hemoglobin antibodies for flowcytometry analysis. Data show that β1-selective AMPK activators and pan-AMPK activators induced fetal hemoglobin in CD34+ cells compared to control (Vehicle as DMSO), increasing the frequency of F-cells (FIG. 6A) with a 1.5-fold change (FIG. 6B).

### Example 7—AMPK Phosphorylation by “Compound 1” Promotes Human Macrophage Polarization to an Anti-Inflammatory Functional Phenotype In Vitro

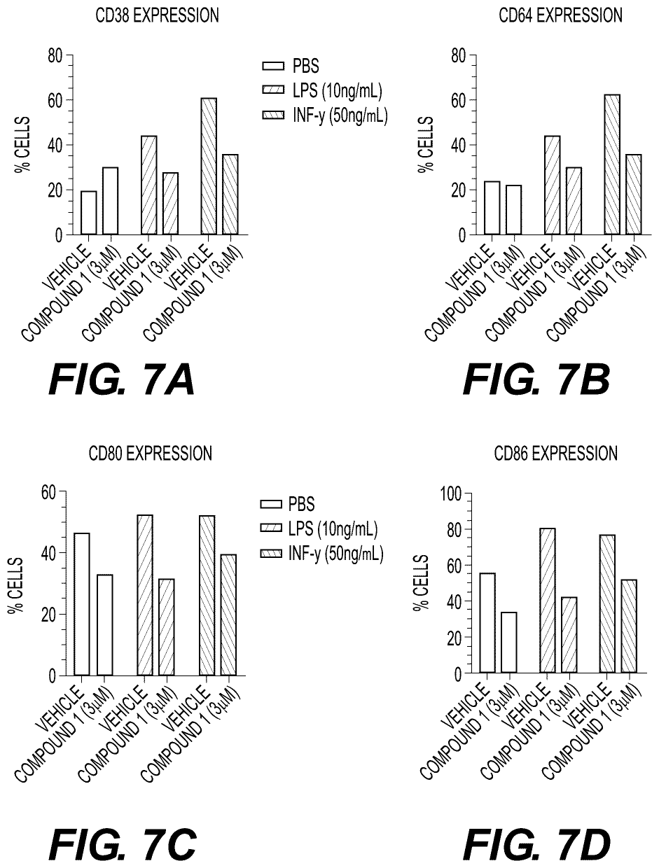

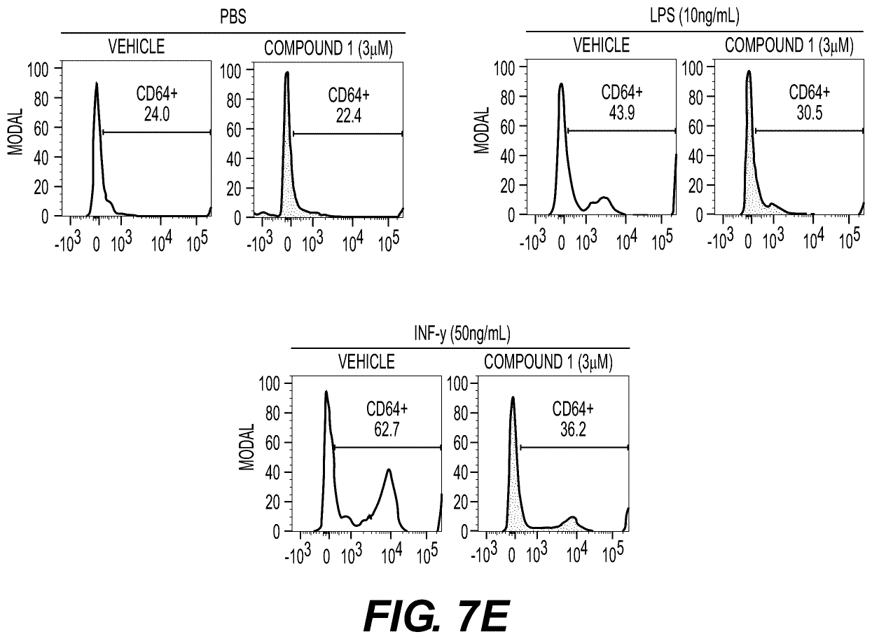

To evaluate the effect of AMPK activation by “Compound 1” on macrophage polarization to pro-inflammatory M1 phenotype, monocytes and macrophages were isolated from total blood collected from a healthy donor by performing a magnetic positive selection for CD14+ cells with magnetic beads (Miltenyi Biotec). M1 macrophages were induced by stimulation with M-CSF (50 ng/mL) for 6 days, then activated by IFN-7 or LPS for 24 h, fixed, stained for M1 polarization markers CD38, CD64, CD86 and CD80 and acquired by flowcytometry. Data shows that activation of AMPK by “Compound 1” in M1-polarized macrophages decreased the expression of proinflammatory M1 markers CD38 (FIG. 7A), CD64 (FIG. 7B), CD80 (FIG. 7C) and CD86 (FIG. 7D).

### Example 8—AMPK Target Engagement in Human CD34+ and Human HUDEP-2 Cells after Exposure to β1-Selective or Pan-AMPK Activators In Vitro

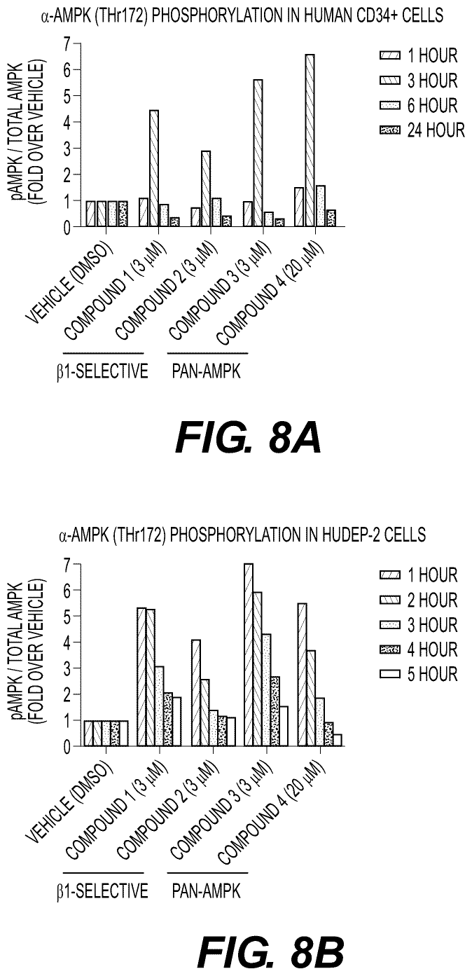

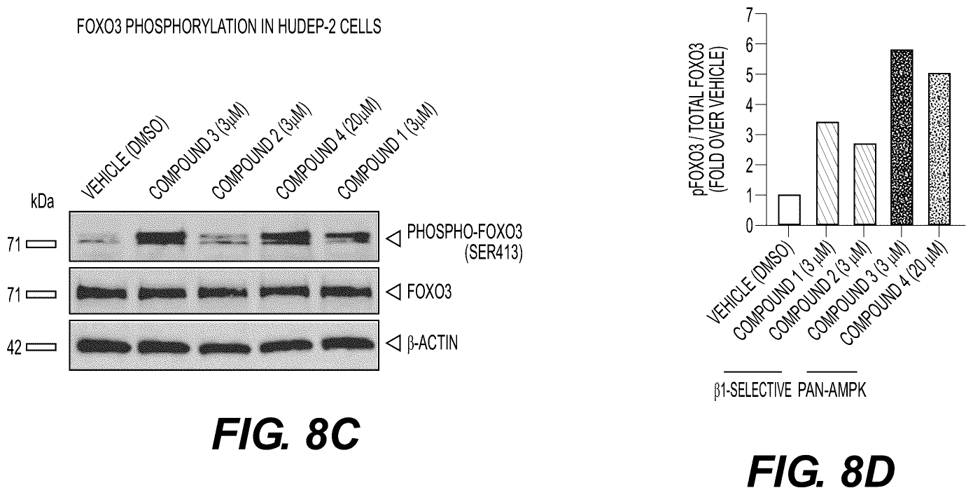

To verify AMPK target engagement by AMPK activators, CD34+ cells from a healthy donor or HUDEP-2 cells (Riken Research Institute, Ibaraki, Japan) were exposed to β1-selective AMPK activators “Compound 1” and “Compound 2”, or to pan-AMPK activators “Compound 3” and “Compound 4” at the indicated doses (μM), harvested and lysed at the indicated time points. Phosphorylation of AMPK at threonine 172 (Thr172) on the α-subunit of AMPK was assessed by HTRF using the Alpha SureFire Ultra Multiplex p-AMPKα1/2 (Thr172)+Total AMPKα1/2 Assay Kit from Perkin Elmer as a target engagement assay. The assay kit contains antibodies, coupled with fluorophore Europium, which recognize the phospho-Thr172 epitope and a distal epitope on α-AMPK of human or mouse AMPK. The kit also contains antibodies coupled with the fluorophore Terbium to measure the total levels of AMPK. As to the in vitro study with human CD34+ cells, cells were collected on Day 11 of differentiation then exposed to β1-selective AMPK activators “Compound 1” and “Compound 2”, or to pan-AMPK activators “Compound 3” and “Compound 4”. As to the human HUDEP-2 cells line, cells were undifferentiated during exposure to AMPK activators. According to the time course, cells were collected and lysed using RIPA buffer mixed with a phosphatase and protease inhibitors cocktail (Thermofisher). Phospho-AMPK signal was divided by total AMPK signal and the ratio was normalized to the total protein concentration. Samples protein concentrations were measured with the Pierce BSA Protein Assay (ThermoFisher). Data show a peak of signal at 3 h after exposure to AMPK activators in CD34+ cells (FIG. 8A), and at 1 h after exposure in HUDEP-2 cells (FIG. 8B), confirming target engagement in AMPK when cells are treated with AMPK activators.

Furthermore, AMPK downstream pathway activation was verified by measuring phosphorylation of FOXO3, a direct target of activated AMPK. Total cell lysates from human erythroid HUDEP-2 cells were generated and the total protein concentration was determined using a Bradford protein assay (ThermoFisher Scientific). Reduced and denaturated protein (40 μg) was loaded and separated by SDS-PAGE (12% gel), blotted on nitrocellulose membranes (BioRad) and finally incubated with FOXO3, Phospho-FOXO3 (Ser413) and p-actin antibodies (Cell Signaling). The β-actin antibody served as an internal control. Immunoreactive proteins were visualized by using an ECL® (enhanced chemiluminescence) detection system (BioRad). Optical density was measured with ImageJ software (National Institutes of Health, Bethesda, MD) and the ratio of phospho-FOXO3 to total FOXO3 was calculated, normalized on β-actin, and plotted based on the densitometry measurements. Blot and quantification confirm the phosphorylation of FOXO3 at Serine 413 in HUDEP-2 cells exposed to AMPK activators, confirming the upstream activation of AMPK due to exposure to AMPK activators (FIGS. 8C and 8D).

### Example 9—AMPK Activation and Induction of Fetal Hemoglobin HbF by “Compound 1” In Vivo in Townes SCD Mice Bone Marrow

To confirm the activation of AMPK and subsequent human fetal hemoglobin induction in mice bone marrow by “Compound 1”, in vivo studies were realized in the sickle cell mouse model Townes mice. These mice express the genes for human HbS (homozygous HbSS), express the physiopathological phenotype of SCD and are called “HbSS-Townes mice”, whereas the control Townes mice express the genes for human HbA without the sickle mutation (homozygous HbAA), are healthy, and are called “HbAA-Townes mice” (Jackson Laboratory). These control HbAA-Townes mice were created by replacing the murine globin genes with human α-globin gene (genotype: Hba hα/hα) and linked human βA− and fetal Aγ-globins (genotype: Hbb hAγβA/hAγβA), whereas HbSS-Townes mice were created by replacing the murine adult α-globin genes with the human α-globin gene (genotype: Hba hα/hα), and the murine adult β-globin genes were replaced with human sickle βS− and fetal Aγ-globin gene fragments linked together (genotype: Hbb hAγβS/hAγβS). All animal studies conformed to the guidelines of the Sanofi IACUC.

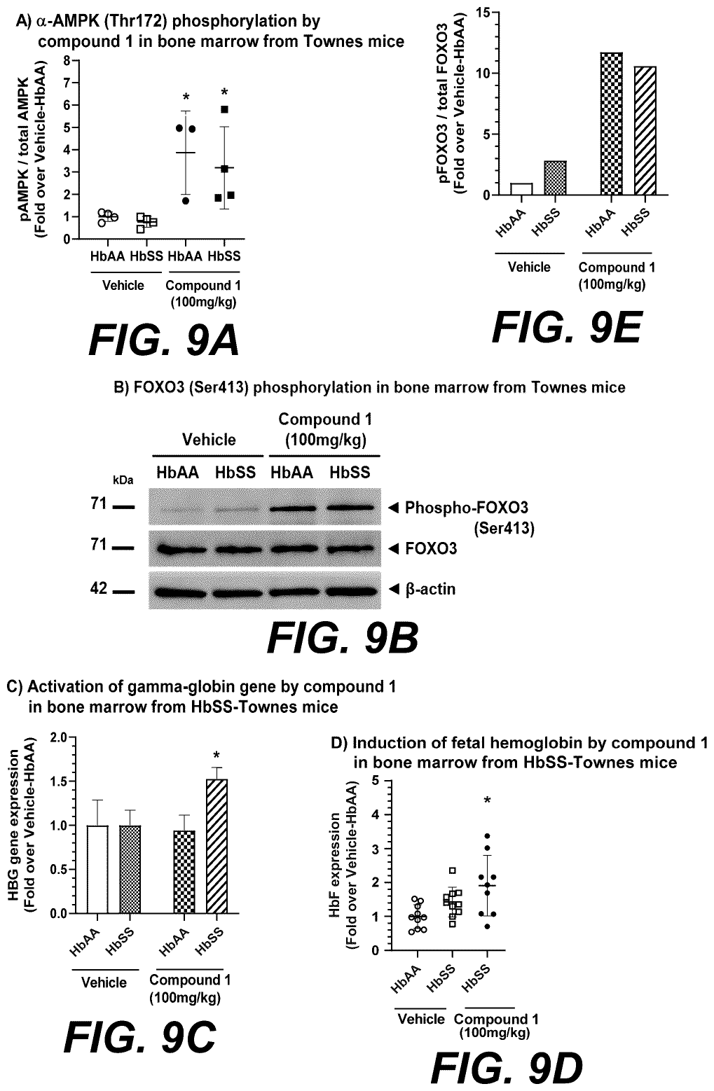

In a 2-day study, mice were given a dose of “Compound 1” at 100 mg/kg per day and by oral gavage in vehicle (0.5% methylcellulose and 0.1% Tween 80). At 2 hours after the last dose on day 2, mice were euthanized and both bone marrow and kidney tissues were collected for protein analysis and measurement of phosphorylation of AMPK for target engagement assessment (Alpha SureFire kit). “Compound 1” increased α-AMPK phosphorylation in the kidneys of Townes HbSS mice (data not shown), in a similar manner as previous rodent studies in rat (Salatto, et al., J. Pharmacol Exp Ther., 2017, 361(2), pp. 303-311). When phosphorylation of α-AMPK in bone marrow cells from HbSS mice and HbAA mice was measured, “Compound 1” exposure resulted in a significant increase in α-AMPK phosphorylation at Thr172 (FIG. 9A). Furthermore, to verify the activation of AMPK downstream pathway, phosphorylation of FOXO3—a direct target of activated AMPK—was measured in mice bone marrow. FOXO3 and Phospho-FOXO3 (Ser413) protein expression level was assessed by Western Blot. The β-actin antibody served as the internal control. Immunoreactive proteins were visualized by using an ECL® (enhanced chemiluminescence) detection system (BioRad), optical density was measured with ImageJ software (National Institutes of Health, Bethesda, MD), and the ratio of phospho-FOXO3 to total FOXO3 was calculated, normalized on β-actin signal, and plotted based on the densitometry measurements. Data confirmed the activation of FOXO3 in bone marrow when Townes-mice are treated with “Compound 1” as shown by the increase of Phospho-FOXO3 (Ser413) in “Compound 1”-treated mice, compared to control (mice treated with Vehicle) (FIGS. 9B and 9C).

Moreover, to study the effect of “Compound 1” on fetal hemoglobin gene expression in bone marrow from Townes mice at the transcriptomic level, quantitative real-time PCR (qRT-PCR) was realized to measure the quantity of mRNA gamma-globin in bone marrow. Total RNA from Townes mouse bone marrow cells was prepared using a RNeasy Mini kit (Qiagen). A quantity of 1 μg of mRNA was reverse transcribed using an iVILO Retro Transcription kit (ThermoFisher). 50 ng of the resulting cDNA was amplified by Taqman amplification in a QuantStudio thermocycler (Life Technologies) using HBG (human 7-globin primers) as gene of interest, and GAPDH (mouse gapdh primers) as a housekeeping gene (Life Technologies). The delta Ct was calculated and difference in mRNA expression was expressed as a fold-change to vehicle condition. Data shows that after 2 days of exposure to “Compound 1”, the mRNA for human HbF (human 7-globin) was increase by 1.5-fold in the HbSS mice, but not in the HbAA mice (FIG. 9D). This reflects the well-known increased rate of erythropoiesis as evidenced by higher levels of reticulocytes in the SCD Townes HbSS mice (45%) versus the control Townes HbAA mice (5%).

Finally, the human fetal hemoglobin protein expression in bone marrow from Townes mice has been measured by flowcytometry after 15-day exposure to “Compound 1” at 100 mg/kg (PO, QD) in a follow-up in vivo chronic study. Data shows an increase of fetal hemoglobin protein expression in bone marrow from Townes mice treated with “Compound 1” compared to control (HbSS mice treated with vehicle) (FIG. 9E).

### Example 10—AMPK Activation by “Compound 1” Decreases Reactive Oxidative Species In Vivo in Bone Marrow from Townes SCD Mice

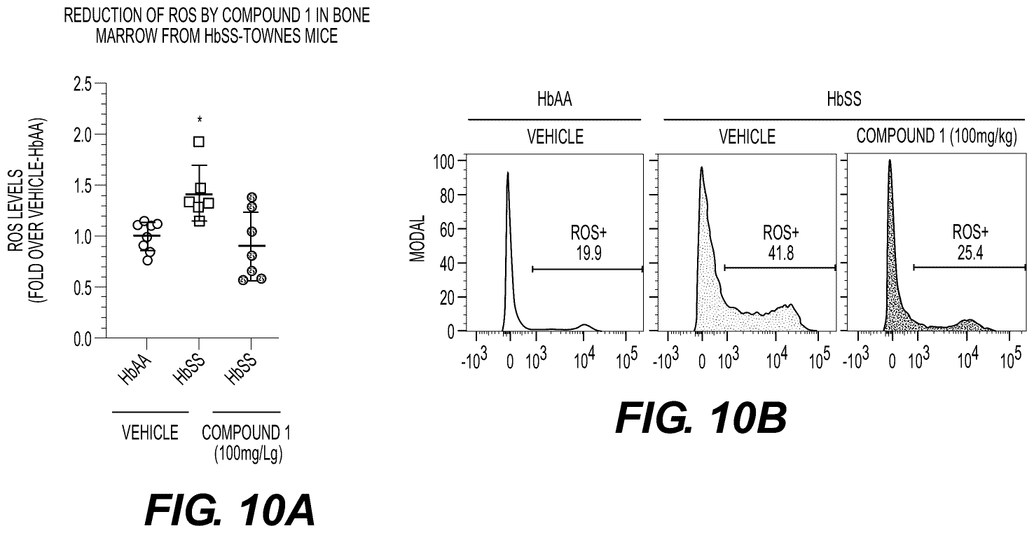

To assess the effect of AMPK activation by “Compound 1” on the oxidative stress process occurring in sickle cell pathophysiology, bone marrow from Townes mice treated for 15 days with “Compound 1” at 100 mg/kg (PO, QD) was isolated, cells were stained with a Reactive Oxidative Species (ROS) dye (Abcam) and signal was acquired by flowcytometry. Results show that “Compound 1” leads to a decrease of ROS in bone marrow compared to control (HbSS mice treated with vehicle) (FIGS. 10A and 10B).

### Example 11—AMPK Activation by “Compound 1” Activates the Nrf2-Oxidative Stress Response Pathway in Human CD34+ Cells In Vitro

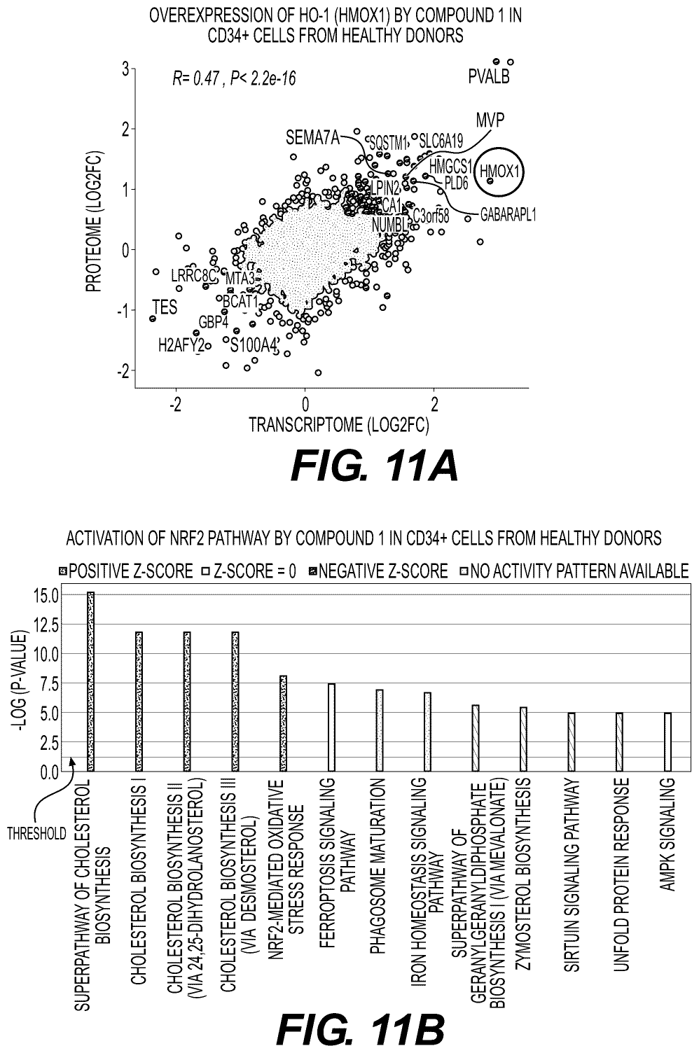

To study the effect of AMPK activation by “Compound 1” in the transcriptome and proteome of human CD34+ cells, a transcriptomic and a proteomic analysis have been performed. As to the transcriptomic analysis, human CD34+ cells from 3 independent healthy donors were cultured and differentiated for 14 days in presence of “Compound 1”. At day 14, RNA was extracted with Trizol reagent (Invitrogen). DNase treatment, RNA integrity and quantification, libraries generation and sequencing (Illumina HiSeq platforms HiSeq 2500) were realized according to Genewiz protocol (South Plainfield, NJ). Concerning the proteomic analysis, proteins were extracted from the same CD34+ cells used for the transcriptomic analysis. Samples preparation, proteins digestion, peptides TMT labeling and proteomic analysis were performed according to IQ Proteomics (Cambridge, MA). Bioinformatics analysis have been conducted to determine the differential expression of mRNA and proteins, and a correlation study using the differential expression from the transcriptomic analysis and the differential expression from the proteomics analysis has been run. The results of that correlation study shows that the proteins HO-1—encoded by HMOX1 gene—and SQSTM1 are upregulated in CD34+ cells treated with “Compound 1” compared to control (DMSO-treated cells) (FIG. 11A). Moreover, an IPA analysis (Ingenuity Pathway Analysis, Qiagen) performed on the proteomics data reveals that the Nrf2-antioxidant response element signaling pathway is activated in CD34+ cells treated with “Compound 1” (FIG. 11B). SQSTM1 is known to activate Nrf2 by inhibiting Keapl, and Nrf2 pathway is known to induce HO-1 for antioxidative function in cells.

### Example 12—Toxicological Study with “Compound 1” In Vivo in Rats does not Modify Hematological Erythroid Parameters

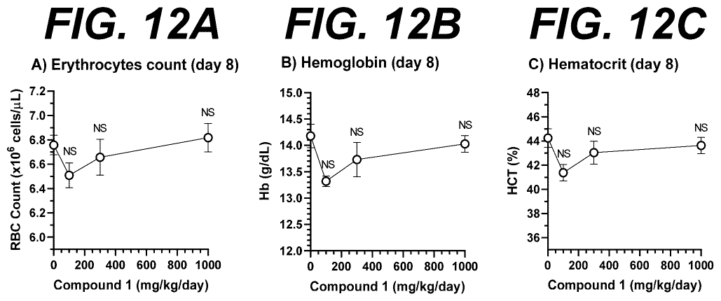

“Compound 1” was administered once daily (QD) to Crl:CD (Sprague Dawley) rats for one week (7 days) by oral gavage (PO, 10 mL/kg, in 0.5% methylcellulose aqueous solution prepared as a suspension). The dose levels for “Compound 1 were 0, 100, 300 and 1000 mg/kg/day. On day 8, blood samples were drawn and complete blood counts (CBC) were tested on a hematology analyzer to determine red blood cell counts (10⁶ RBC/μL), hemoglobin (Hb, g/dL) and hematocrit (HCT, %). Daily exposure to “Compound” 1 does not reduce RBC (FIG. 12A), hemoglobin (FIG. 12B) and hematocrit (FIG. 12C).

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

1. A method of treating or preventing a β-hemoglobinopathy, the method comprising administering to a patient in need thereof a therapeutically effective amount of a β1-AMPK activator.

2. A method of increasing HbF expression, the method comprising administering to a patient in need thereof a therapeutically effective amount of a β1-AMPK activator.

3. A method of decreasing inflammation or decreasing oxidative stress in β-hemoglobinopathy, the method comprising administering to a patient in need thereof a therapeutically effective amount of a β31-AMPK activator.

4. (canceled)

5. The method according to claim 1, wherein the β1-AMPK activator is a compound of Formula (I): 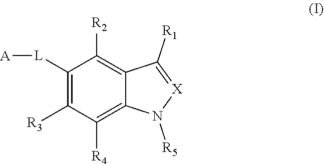
or a pharmaceutically acceptable salt thereof, wherein
X is N or CH;
R₁ is —C(O)OR_{A}, —C(O)NR_{B}R_{C}, —S(O₂)OR_{A}, —S(O₂)NHC(O)R_{D},
5-oxo-4,5-dihydro-1,2,4-oxadiazol-3-yl, or 1H-tetrazol-5-yl;
R_{A} is H or (C₁-C₆)alkyl;
R_{B} and R_{C} are independently H, (C₁-C₆)alkyl, or —S(O₂)R_{D};
R_{D} is (C₁-C₆)alkyl, —CF₃, or phenyl, wherein the phenyl is optionally substituted with 1, 2, 3, 4, or 5 substituents that are independently (C₁-C₆)alkoxy, (C₁-C₆)alkyl, cyano, halogen, halo(C₁-C₆)alkoxy, halo(C₁-C₆)alkyl, hydroxy, mercapto, nitro, or NR_{E}R_{F};
R_{E} and R_{F} are independently H or (C₁-C₆)alkyl;
R₂, R₃, and R₄ are independently H, (C₁-C₆)alkoxy, (C₁-C₆)alkyl, (C₁-C₆)alkylthio, carboxy, cyano, halogen, halo(C₁-C₆)alkoxy, halo(C₁-C₆)alkyl, hydroxy,
hydroxy(C₁-C₈)alkyl, mercapto, nitro, —NR_{G}R_{H} or (NR_{G}R_{H})carbonyl;
R_{G} and R_{H} are independently H, (C₁-C₆)alkyl, or (C₁-C₆)alkylcarbonyl;
R₅ is H or (C₁-C₆)alkyl;
L is a bond, O, S, NRA, (C₁-C₆)alkylene, (C₂-C₆)alkenylene, or (C₂-C₆)alkynylene;
A is phenyl, 2,3-dihydrobenzo[b][1,4]dioxinyl, 2,3-dihydrobenzofuranyl,
2,3-dihydro-1H-indenyl, imidazolyl, pyrazinyl, pyrazolyl, pyridinyl, pyrimidinyl, or thiazolyl, wherein each is optionally substituted with 1, 2, 3, 4, or 5 substituents that are independently (C₁-C₆)alkoxy, (C₁-C₆)alkoxy(C₁-C₆)alkoxy, (C₁-C₆)alkoxy(C₁-C₆)alkyl, (C₁-C₆)alkoxycarbonyl, (C₁-C₆)alkyl, (C₁-C₆)alkylcarbonyl, (C₁-C₆)alkylthio, aryl, aryl(C₁-C₆)alkoxy, aryl(C₁-C₆)alkyl, arylcarbonyl, aryloxy, carboxy,
carboxy(C₁-C₆)alkoxy, carboxy(C₁-C₆)alkyl, cyano, (C₃-C₈)cycloalkyl,
(C₃-C₈)cycloalkyl(C₁-C₆)alkoxy, (C₃-C₈)cycloalkyl(C₁-C₆)alkyl, (C₃-C₈)cycloalkylcarbonyl, (C₃-C₈)cycloalkyloxy, halogen, halo(C₁-C₆)alkoxy, halo(C₁-C₆)alkyl, heteroaryl, heteroaryl(C₁-C₆)alkoxy, heteroaryl(C₁-C₆)alkyl, heteroarylcarbonyl, heteroaryloxy, (C₃-C₇)heterocycle, (C₃-C₇)heterocycle(C₁-C₆)alkoxy, (C₃-C₇)heterocycle(C₁-C₆)alkyl, (C₃-C₇)heterocyclecarbonyl, (C₃-C₇)heterocyclecarbonyl(C₁-C₆)alkyl,
(C₃-C₇)heterocycleoxy, hydroxy, hydroxy(C₁-C₆)alkoxy, hydroxy(C₁-C₆)alkyl, mercapto, nitro, —NR_{J}R_{K}, (NR_{J}R_{K})carbonyl, —NR_{M}R_{N}, —NR_{M}R_{N}(C₁-C₆)alkoxy, (NR_{M}R_{N})carbonyl, (NR_{M}R_{N})carbonyl(C₁-C₆)alkyl, or (NR_{M}R_{N})carbonyl(C₁-C₆)alkoxy; wherein the aryl, aryl(C₁-C₆)alkoxy, aryl(C₁-C₆)alkyl, arylcarbonyl, and aryloxy are optionally substituted with 1, 2, 3, 4, or 5 substituents that are independently (C₁-C₆)alkoxy,
(C₁-C₆)alkoxycarbonyl, (C₁-C₆)alkyl, (C₁-C₆)alkylcarbonyl, (C₁-C₆)alkylthio, carboxy, cyano, halogen, halo(C₁-C₆)alkoxy, halo(C₁-C₆)alkyl, hydroxy, hydroxy(C₁-C₆)alkyl, mercapto, nitro, —NR_{M}R_{N}, or (NR_{M}R_{N})carbonyl; wherein the halo(C₁-C₆)alkyl is optionally substituted with 1 or 2 hydroxy groups; wherein the (C₃-C₈)cycloalkyl,
(C₃-C₈)cycloalkyl(C₁-C₆)alkoxy, (C₃-C₈)cycloalkyl(C₁-C₆)alkyl, (C₃-C₈)cycloalkylcarbonyl, and
(C₃-C₈)cycloalkyloxy are optionally substituted with 1, 2, or 3 substituents that are independently (C₁-C₆)alkoxy, (C₁-C₆)alkoxycarbonyl, (C₁-C₆)alkyl, (C₁-C₆)alkylcarbonyl, (C₁-C₆)alkylthio, carboxy, cyano, halogen, halo(C₁-C₆)alkoxy, halo(C₁-C₆)alkyl, hydroxy, hydroxy(C₁-C₆)alkyl, mercapto, nitro, —NR_{M}R_{N}, or (NR_{M}R_{N})carbonyl; wherein the heteroaryl, heteroaryl(C₁-C₆)alkoxy, heteroaryl(C₁-C₆)alkyl, heteroarylcarbonyl, and heteroaryloxy, are optionally substituted with 1, 2, or 3 substituents that are
independently (C₁-C₆)alkoxy, (C₁-C₆)alkoxycarbonyl, (C₁-C₆)alkyl, (C₁-C₆)alkylcarbonyl, (C₁-C₆)alkylthio, carboxy, cyano, halogen, halo(C₁-C₆)alkoxy, halo(C₁-C₆)alkyl, hydroxy, hydroxy(C₁-C₆)alkyl, mercapto, nitro, —NR_{M}R_{N}, or (NR_{M}R_{N})carbonyl; and wherein the (C₃-C₇)heterocycle, (C₃-C₇)heterocycle(C₁-C₆)alkoxy, (C₃-C₇)heterocycle(C₁-C₆)alkyl, (C₃-C₇)heterocyclecarbonyl, (C₃-C₇)heterocyclecarbonyl(C₁-C₆)alkyl, and
(C₃-C₇)heterocycleoxy, are optionally substituted with 1, 2, or 3 substituents that are independently (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylsulfonyl, (C₁-C₆)alkylthio, carboxy, cyano, halogen, halo(C₁-C₆)alkoxy, halo(C₁-C₆)alkyl, hydroxy, hydroxy(C₁-C₆)alkyl, mercapto, nitro, —NR_{M}R_{N}, (NR_{M}R_{N})carbonyl, or oxo;
R_{J} and R_{K} are independently H or (C₁-C₆)alkyl; and
R_{M} and R_{N} are independently H, (C₁-C₆)alkyl, or (C₁-C₆)alkylcarbonyl; or R_{M} and
R_{N} together with the nitrogen they are attached to form a 3 to 8 membered ring;
provided that Formula (I) does not encompass
5-(4-bromophenyl)-1H-indole-3-carboxamide;
5-(2′,6′-dihydroxy-[1,1′-biphenyl]-4-yl)-1H-indole-3-carboxamide; and
5-(2′,6′-dimethoxy-[1,1]biphenyl]-4-yl)-1H-indole-3-carboxamide.

6. The method according to claim 1, wherein the β1-AMPK activator is a compound of Formula (I): 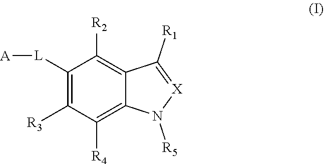
or a pharmaceutically acceptable salt thereof, wherein
X is N or CH;
L is a bond, O, S, NRA, (C₁-C₆)alkylene, (C₂-C₆)alkenylene, or C₂-C₆ alkynlene; 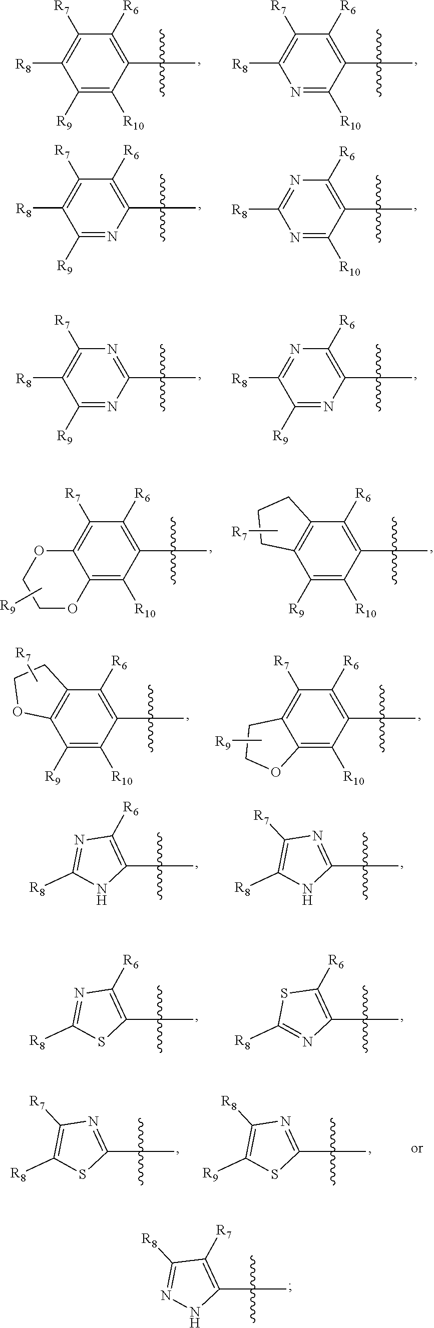
Ri is —C(O)OR_{A}, —C(O)NR_{B}R_{C}, —S(O₂)OR_{A}, —S(O₂)NHC(O)R_{D},
5-oxo-4,5-dihydro-1,2,4-oxadiazol-3-yl, or 1H-tetrazol-5-yl;
R_{A} is H or (C₁-C₆)alkyl;
R_{B} and R_{C} are independently H, (C₁-C₆)alkyl, or —S(O₂)R_{D};
R_{D} is (C₁-C₆)alkyl, —CF₃, or phenyl, wherein the phenyl is optionally substituted with 1, 2, 3, 4, or 5 substituents that are independently (C₁-C₆)alkoxy, (C₁-C₆)alkyl, cyano, halogen, halo(C₁-C₆)alkoxy, halo(C₁-C₆)alkyl, hydroxy, mercapto, nitro, or NR_{E}R_{F};
R_{E} and R_{F} are independently H or (C₁-C₆)alkyl;
R₂, R₃, and R₄ are independently H, (C₁-C₆)alkoxy, (C₁-C₆)alkyl, (C₁-C₆)alkylthio, carboxy, cyano, halogen, halo(C₁-C₆)alkoxy, halo(C₁-C₆)alkyl, hydroxy,
hydroxy(C₁-C₈)alkyl, mercapto, nitro, —NR_{G}R_{H}, or (NR_{G}R_{H})carbonyl;
R_{G} and R_{H} are independently H, (C₁-C₆) alkyl, or (C₁-C₆) alkylcarbonyl;
R₅ is H or (C₁-C₆)alkyl;
R₆, R₇, R₉, and R₁₀ are independently H, (C₁-C₆)alkoxy, (C₁-C₆)alkoxycarbonyl, (C₁-C₆)alkyl, (C₁-C₆)alkylcarbonyl, (C₁-C₆)alkylthio, carboxy, cyano, halogen,
halo(C₁-C₆)alkoxy, halo(C₁-C₆)alkyl, hydroxy, hydroxy(C₁-C₆)alkyl, mercapto, nitro, —NR_{J}R_{K}, or (NR_{J}R_{K})carbonyl;
R_{J} and R_{K} are independently H or (CrC₆)alkyl;
R₈ is H, (C₁-C₆)alkoxy, (C₁-C₆)alkoxy(C₁-C₆)alkoxy, (C₁-C₆)alkoxy(C₁-C₆)alkyl, (C₁-C₆)alkoxycarbonyl, (C₁-C₆)alkyl, (C₁-C₆)alkylcarbonyl, (C₁-C₆)alkylthio, aryl, aryl(C₁-C₆)alkoxy, aryl(C₁-C₆)alkyl, arylcarbonyl, aryloxy, carboxy,
carboxy(C₁-C₆)alkoxy, carboxy(C₁-C₆)alkyl, cyano, (C₃-C₈)cycloalkyl,
(C₃-C₈)cycloalkyl(C₁-C₆)alkoxy, (C₃-C₈)cycloalkyl(C₁-C₆)alkyl, (C₃-C₈)cycloalkylcarbonyl, (C₃-C₈)cycloalkyloxy, halogen, halo(C₁-C₆)alkoxy, halo(C₁-C₆)alkyl, heteroaryl, heteroaryl(C₁-C₆)alkoxy, heteroaryl(C₁-C₆)alkyl, heteroarylcarbonyl, heteroaryloxy, (C₃-C₇)heterocycle, (C₃-C₇)heterocycle(C₁-C₆)alkoxy, (C₃-C₇)heterocycle(C₁-C₆)alkyl, (C₃-C₇)heterocyclecarbonyl, (C₃-C₇)heterocyclecarbonyl(C₁-C₆)alkyl, (C₃-C₇)heterocycleoxy, hydroxy, hydroxy(C₁-C₆)alkoxy, hydroxy(C₁-C₆)alkyl, mercapto, nitro, —NR_{M}R_{N}, —NR_{M}R_{N}(C₁-C₆)alkoxy, (NR_{M}R_{N})carbonyl, (NR_{M}R_{N})carbonyl(C₁-C₆)alkyl, or (NR_{M}R_{N})carbonyl(C₁-C₆)alkoxy; wherein the aryl, aryl(C₁-C₆)alkoxy, aryl(C₁-C₆)alkyl, arylcarbonyl, and aryloxy are optionally substituted with 1, 2, 3, 4, or 5 substituents that are independently (C₁-C₆)alkoxy, (C₁-C₆)alkoxycarbonyl, (C₁-C₆)alkyl, (C₁-C₆)alkylcarbonyl, (C₁-C₆)alkylthio, carboxy, cyano, halogen, halo(C₁-C₆)alkoxy, halo(C₁-C₆)alkyl, hydroxy, hydroxy(C₁-C₆)alkyl, mercapto, nitro, —NR_{M}R_{N}, or
(NR_{M}R_{N})carbonyl; wherein the halo(C₁-C₆)alkyl is optionally substituted with 1 or 2 hydroxy groups; wherein the (C₃-C₈)cycloalkyl, (C₃-C₈)cycloalkyl(C₁-C₆)alkoxy,
(C₃-C₈)cycloalkyl(C₁-C₆)alkyl, (C₃-C₈)cycloalkylcarbonyl, and (C₃-C₈)cycloalkyloxy are optionally substituted with 1, 2, or 3 substituents that are independently (C₁-C₆)alkoxy, (C₁-C₆)alkoxycarbonyl, (C₁-C₆)alkyl, (C₁-C₆)alkylcarbonyl, (C₁-C₆)alkylthio, carboxy, cyano, halogen, halo(C₁-C₆)alkoxy, halo(C₁-C₆)alkyl, hydroxy, hydroxy(C₁-C₆)alkyl, mercapto, nitro, —NR_{M}R_{N}, or (NR_{M}R_{N})carbonyl; wherein the heteroaryl,
heteroaryl(C₁-C₆)alkoxy, heteroaryl(C₁-C₆)alkyl, heteroarylcarbonyl, and heteroaryloxy, are optionally substituted with 1, 2, or 3 substituents that are independently (d-C₆)alkoxy, (d-C₆)alkoxycarbonyl, (C₁-C₆)alkyl, (C₁-C₆)alkylcarbonyl,
(C₁-C₆)alkylthio, carboxy, cyano, halogen, halo(C₁-C₆)alkoxy, halo(C₁-C₆)alkyl, hydroxy, hydroxy(C₁-C₆)alkyl, mercapto, nitro, —NR_{M}R_{N}, or (NR_{M}R_{N})carbonyl; and wherein the (C₃-C₇)heterocycle, (C₃-C₇)heterocycle(C₁-C₆)alkoxy, (C₃-C₇)heterocycle(C₁-C₆)alkyl, (C₃-C₇)heterocyclecarbonyl, (C₃-C₇)heterocyclecarbonyl(C₁-C₆)alkyl, and
(C₃-C₇)heterocycleoxy, are optionally substituted with 1, 2, or 3 substituents that are independently (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylsulfonyl, (C₁-C₆)alkylthio, carboxy, cyano, halogen, halo(C₁-C₆)alkoxy, halo(C₁-C₆)alkyl, hydroxy, hydroxy(C₁-C₆)alkyl, mercapto, nitro, —NR_{M}R_{N}, (NR_{M}R_{N})carbonyl, or oxo; and
R_{M} and R_{N} are independently H, (C₁-C₆)alkyl, or (C₁-C₆)alkylcarbonyl; or R_{M} and R_{N} together with the nitrogen they are attached to form a 3 to 8 membered ring;
provided that Formula (I) does not encompass:
5-(4-bromophenyl)-1H-indole-3-carboxamide;
5-(2′,6′-dihydroxy-[1,1′-biphenyl]-4-yl)-1H-indole-3-carboxamide; and
5-(2′,6′-dimethoxy-[1,1]biphenyl]-4-yl)-1H-indole-3-carboxamide.

7. The method according to claim 1, wherein the β1-AMPK activator is a compound of Formula (I), or a pharmaceutically acceptable salt thereof, wherein X is N or CH; L is a bond or —C₆)alkynylene;
A is 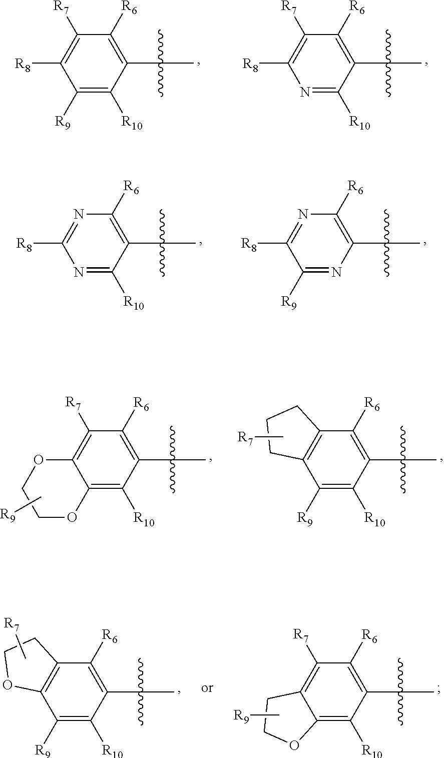
R₁ is —C(O)OR_{A}, —C(O)R_{B}R_{C}, —S(O₂)OR_{A};
R_{A} is H;
R_{B} and R_{C} are independently H or —S(O₂)R_{D};
R_{D} is (C₁-C₆)alkyl, —CF₃, or phenyl;
R₂, R₃, and R₄ are independently H, (C₁-C₆)alkyl, cyano, or halogen;
R₅ is H;
R₆, R₇, R₉, and R₁₀ are independently H, (C₁-C₆)alkoxy, (C₁-C₆)alkyl, cyano, halogen, hydroxy, or hydroxy(C₁-C₆)alkyl:
R₈ is H, (C₁-C₆)alkoxy, (C₁-C₆)alkoxy(C₁-C₆)alkyl, (C₁-C₆)alkyl, (C₁-C₆)alkylcarbonyl, aryl, carboxy(C₁-C₆)alkoxy, (C₃-C₈)cycloalkyl, (C₃-C₈)cycloalkyl(C₁-C₆)alkyl, (C₃-C₈)cycloalkyloxy, halo(C₁-C₆)alkyl, heteroaryl(C₁-C₆)alkoxy, (C₃-C₇)heterocycle, (C₃-C₇)heterocycle(C₁-C₆)alkoxy, (C₃-C₇)heterocyclecarbonyl(C₁-C₆)alkyl, (C₃-C₇)heterocycleoxy, hydroxy(C₁-C₆)alkoxy, hydroxy(C₁-C₆)alkyl, —NR_{M}R_{N}, (NR_{M}R_{N})carbonyl(C₁-C₆)alkyl, or (NR_{M}R_{N})carbonyl(C₁-C₆)alkoxy; wherein the aryl is optionally substituted with 1 substituent that is (C₁-C₆)alkoxy or hydroxy; wherein the halo(C₁-C₆)alkyl is optionally with 1 hydroxy group;
wherein the (C₃-C₈)cycloalkyl, (C₃-C₈)cycloalkyl(C₁-C₆)alkyl, and (C₃-C₈)cycloalkyloxy are optionally substituted with 1 substituent that is carboxy, hydroxy, hydroxy(C₁-C₆)alkyl, or (NR_{M}R_{N})carbonyl; and wherein the (C₃-C₇)heterocycle and (C₃-C₇)heterocycle(C₁-C₆)alkoxy are optionally substituted with 1 substituent that is (C₁-C₆)alkoxycarbonyl, (C₁-C₆)alkyl, (C₁-C₆)alkylcarbonyl, (C₁-C₆)alkylsulfonyl, hydroxy, hydroxy(C₁-C₆)alkyl, or oxo; and R_{M} and R_{N} are independently H, (C₁-C₆)alkyl, or (C₁-C₆)alkylcarbonyl; or R_{M} and R_{N} together with the nitrogen they are attached to form a 3 to 8 membered ring.

8. The method according to claim 1, wherein the β1-AMPK activator is a compound of Formula (II): 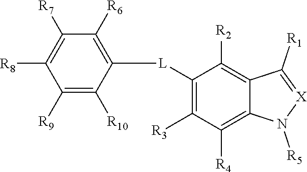
or a pharmaceutically acceptable salt thereof, wherein:
X is N or CH;
L is a bond, O, S, NRA, (C₁-C₆)alkylene, (C₂-C₆)alkenylene, or (C₂-C₆)alkynylene;
R₁ is —C(O)OR_{A}, —C(O)NR_{B}R_{C}, —S(O₂)OR_{A}, —S(O₂)NHC(O)R_{D}, 5-oxo-4,5-dihydro-1,2,4-oxadiazol-3-yl, or 1H-tetrazol-5-yl;
R_{A} is H or (C₁-C₆)alkyl;
R_{B} and R_{C} are independently H, (C₁-C₆)alkyl, or —S(O₂)R_{D};
R_{D} is (C₁-C₈)alkyl, —CF₃, or phenyl, wherein the phenyl is optionally substituted with 1, 2, 3, 4, or 5 substituents that are independently (C₁-C₆)alkoxy, (C₁-C₆)alkyl, cyano, halogen, halo(C₁-C₆)alkoxy, halo(C₁-C₆)alkyl, hydroxy, mercapto, nitro, or NR_{E}R_{F};
R_{E} and R_{F} are independently H or (C₁-C₆)alkyl;
R₂, R₃, and R₄ are independently H, (C₁-C₆)alkoxy, (C₁-C₆)alkyl, (C₁-C₆)alkylthio, carboxy, cyano, halogen, halo(C₁-C₆)alkoxy, halo(C₁-C₆)alkyl, hydroxy, hydroxy(C₁-C₆)alkyl, mercapto, nitro, —NR_{G}R_{H}, or (NR_{G}R_{H})carbonyl; R_{G} and R_{H} are independently H, (C₁-C₆)alkyl, or (C₁-C₆)alkylcarbonyl; R₅ is H or (C₁-C₆)alkyl;
R₆, R₇, R₉, and R₁₀ are independently H, (C₁-C₆)alkoxy, (C₁-C₆)alkoxycarbonyl, (C₁-C₆)alkyl, (C₁-C₆)alkylcarbonyl, (C₁-C₆)alkylthio, carboxy, cyano, halogen, halo(C₁-C₆)alkoxy, halo(C₁-C₆)alkyl, hydroxy, hydroxy(C₁-C₆)alkyl, mercapto, nitro, —NR_{J}R_{K}, or (NR_{J}R_{K})carbonyl; R_{J} and R_{K} are independently H or (C₁-C₆)alkyl;
R₈ is H, (C₁-C₆)alkoxy, (C₁-C₆)alkoxy(C₁-C₆)alkoxy, (C₁-C₆)alkoxy(C₁-C₆)alkyl, (C₁-C₆)alkoxycarbonyl, (C₁-C₆)alkyl, (C₁-C₆)alkylcarbonyl, (C₁-C₆)alkylthio, aryl, aryl(C₁-C₆)alkoxy, aryl(C₁-C₆)alkyl, arylcarbonyl, aryloxy, carboxy, carboxy(C₁-C₆)alkoxy, carboxy(C₁-C₆)alkyl, cyano, (C₃-C₈)cycloalkyl, (C₃-C₈)cycloalkyl(C₁-C₆)alkoxy, (C₃-C₈)cycloalkyl(C₁-C₆)alkyl, (C₃-C₈)cycloalkylcarbonyl, (C₃-C₈)cycloalkyloxy, halogen, halo(C₁-C₆)alkoxy, halo(C₁-C₆)alkyl, heteroaryl, heteroaryl(C₁-C₆)alkoxy, heteroaryl(C₁-C₆)alkyl, heteroarylcarbonyl, heteroaryloxy, (C₃-C₇)heterocycle, (C₃-C₇)heterocycle(C₁-C₆)alkoxy, (C₃-C₇)heterocycle(C₁-C₆)alkyl, (C₃-C₇)heterocyclecarbonyl(C₃-C₇)heterocyclecarbonyl(C₁-C₆)alkyl, (C₃-C₇)heterocycleoxy, hydroxy, hydroxy(C₁-C₆)alkoxy, hydroxy(C₁-C₆)alkyl, mercapto,nitro, —NR_{M}R_{N}, —NR_{M}R_{N}(C₁-C₆))alkoxy, (NR_{M}R_{N})carbonyl, (NR_{M}R_{N})carbonyl(C₁-C₆)alkyl, or (NR_{M}R_{N})carbonyl(C₁-C₆)alkoxy; wherein the aryl, aryl(C₁-C₆)alkoxy, aryl(C₁-C₆)alkyl, arylcarbonyl, and aryloxy are optionally substituted with 1, 2, 3, 4, or 5 substituents that are independently (C₁-C₆)alkoxy, (C₁-C₆)alkoxycarbonyl, (C₁-C₆)alkyl, (C₁-C₆)alkylcarbonyl, (C₁-C₆)alkylthio, carboxy, cyano, halogen, halo(C₁-C₆)alkoxy, halo(C₁-C₆)alkyl, hydroxy, hydroxy(C₁-C₆)alkyl, mercapto, nitro, —NR_{M}R_{N}, or (NR_{M}R_{N})carbonyl; wherein the halo(C₁-C₆)alkyl is optionally substituted with 1 or 2 hydroxy groups; wherein the (C₃-C₇)cycloalkyl, (C₃-C₇)cycloalkyl(C₁-C₆)alkoxy, (C₃-C₇)cycloalkyl(C₁-C₆)alkyl, (C₃-C₇)cycloalkylcarbonyl, and
(C₃-C₇)cycloalkyloxy are optionally substituted with 1, 2, or 3 substituents that are independently (C₁-C₆)alkoxy, (C₁-C₆)alkoxycarbonyl, (C₁-C₆)alkyl, (C₁-C₆)alkylcarbonyl, (C₁-C₆)alkylthio, carboxy, cyano, halogen, halo(C₁-C₆))alkoxy, halo(C₁-C₆)alkyl, hydroxy, hydroxy(C₁-C₆)alkyl, mercapto, nitro, —NR_{M}R_{N}, or (NR_{M}R_{N})carbonyl; wherein the heteroaryl, heteroaryl(C₁-C₆)alkoxy, heteroaryl(C₁-C₆)alkyl, heteroarylcarbonyl, and heteroaryloxy, are optionally substituted with 1, 2, or 3 substituents that are independently
(C₁-C₆)alkoxy, (C₁-C₆)alkoxycarbonyl, (C₁-C₆)alkyl, (C₁-C₆)alkylcarbonyl,
(C₁-C₆)alkylthio, carboxy, cyano, halogen, halo(C₁-C₆)alkoxy, halo(C₁-C₆))alkyl, hydroxy, hydroxy(C₁-C₆)alkyl, mercapto, nitro, —NR_{M}R_{N}, or (NR_{M}R_{N})carbonyl; and wherein the (C₃-C₇)heterocycle, (C₃-C₇)heterocycle(C₁-C₆)alkoxy, (C₃-C₇)heterocycle(C₁-C₆)alkyl, (C₃-C₇)heterocyclecarbonyl, (C₃-C₇)heterocyclecarbonyl(C₁-C₆)alkyl, and
(C₃-C₇)heterocycleoxy, are optionally substituted with 1, 2, or 3 substituents that are independently (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylsulfonyl, (C₁-C₆)alkylthio, carboxy, cyano, halogen, halo(C₁-C₆)alkoxy, halo(C₁-C₆)alkyl, hydroxy, hydroxy(C₁-C₆))alkyl, mercapto, nitro, —NR_{M}R_{N}, (NR_{M}R_{N})carbonyl, or oxo; and R_{M} and R_{N} are independently H, (C₁-C₆)alkyl, or (C₁-C₆)alkylcarbonyl; and R_{M} and R_{N} together with the nitrogen they are attached to form a 3 to 8 membered ring; provided that Formula (II) does not encompass
5-(4-bromophenyl)-1H-indole-3-carboxamide; 5-(2′,6′-dihydroxy-[1,1′-biphenyl]-4-yl)-1H-indole-3-carboxamide; and 5-(2′,6′-dimethoxy-[1,1]biphenyl]-4-yl)-1H-indole-3-carboxamide.

9. The method according to claim 1, wherein the β1-AMPK activator is a compound of Formula (II): 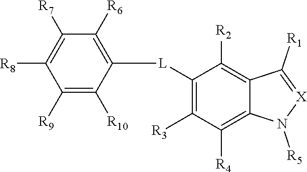
or a pharmaceutically acceptable salt thereof, wherein:
X is CH;
L is a bond;
R₁ is —C(O)OR_{A};
R_{A} is H;
R₂ is H or F;
R₃ is Cl, F, or CN;
R₄ and R₅ are H;
R₆ and R₇ are independently H, F, or methoxy;
R₉ and R₁₀ are H; and
R₈ is (C₃-C₈)cycloalkyl wherein the (C₃-C₈)cycloalkyl
is cyclopropyl or cyclobutyl substituted with hydroxy.

10. The method according to claim 9, wherein the β1-AMPK activator is a compound of Formula (II) above, selected from the group consisting of:
6-chloro-5-[2-fluoro-4-(1-hydroxycyclobutyl)phenyl]11H-indole-3-carboxylic acid;
6-chloro-5-[3-fluoro-4-(1-hydroxycyclobutyl)phenyl]-1H-indole-3-carboxylic acid; and
6-chloro-5-[4-(1-hydroxycyclobutyl)-3-methoxyphenyl]-1H-indole-3-carboxylic acid;
or a pharmaceutically acceptable salt thereof.

11. The method according to claim 1, wherein the compound of formula (I) is 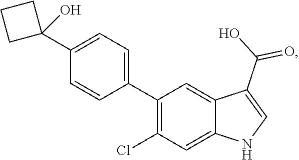 [SMILES: O=C(O)c1c[nH]c2cc(Cl)c(-c3ccc(C4(O)CCC4)cc3)cc12]
or a pharmaceutically acceptable salt thereof.

12. The method according to claim 1, wherein the β1-AMPK activator is 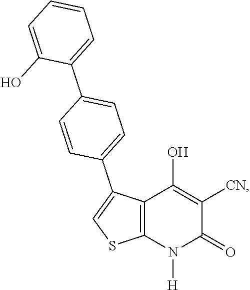 [SMILES: N#Cc1c(O)c2c(-c3ccc(-c4ccccc4O)cc3)csc2[nH]c1=O]
or a pharmaceutically acceptable salt thereof.

13. The method according to claim 1, wherein the β1-AMPK activator is a β1-selective AMPK activator.

14. The method according to claim 13, wherein the β1-selective AMPK activator possesses at least about a 10-fold, about a 50-fold, about a 100-fold, or about a 300-fold selective activation for β1-AMPK relative to β2-AMPK.

15. (canceled)

16. (canceled)

17. (canceled)

18. The method according to claim 13, wherein the β1-selective AMPK activator has an EC₅₀ for the activation of β1-AMPK of about 100 nM or less, about 50 nM or less, or about 10 nM or less.

19. (canceled)

20. (canceled)

21. (canceled)

22. The method according to claim 13, wherein the β1-selective AMPK activator increases the activity of AMPK above the baseline by 50% or more, by 100% or more or by 150% r more.

23. (canceled)

24. (canceled)

25. The method according to claim 1, wherein the p-hemoglobinopathy is sickle cell disease (SCD) or β-thalassemia.

26. (canceled)

27. The method according to claim 1, wherein the patient has an HbS/β⁰ genotype, an HbS/μ⁺ genotype, an HBSC genotype, an HbS/HbE genotype, an HbD Los Angeles genotype, a G-Philadelphia genotype, or an abHbO Arab genotype.

28. The method according to claim 1, wherein the β1-AMPK activator is administered in combination with hydroxyurea.
